(19) United States
(12) Patent Application Publication
(10) Pub. No.: US 2022/0243171 A1
(43) Pub. Date: Aug. 4, 2022

(54) SYSTEMS AND METHODS RELATING TO THREE-DIMENSIONAL (3D) CELL MANUFACTURING

(71) Applicant: University of Florida Research Foundation, Inc., Gainesville, FL (US)

(72) Inventors: Thomas Ettor ANGELINI, Gainesville, FL (US); Cameron D MORLEY, Gainesville, FL (US); Christopher S. O'BRYAN, Gainesville, FL (US); Peter S. MCFETRIDGE, Gainesville, FL (US); Malisa SARNTINORANONT, Gainesville, FL (US); Tanmay P. LELE, Gainesville, FL (US); Naohiro Terada, Nishinomiya (JP)

(21) Appl. No.: 17/622,102

(22) PCT Filed: Jul. 1, 2020
(86) PCT No.: PCT/US2020/040490

§ 371 Date: Dec. 22, 2021

Related U.S. Application Data
(60) Provisional application No. 62/869,308, filed on Jul. 1, 2019.

(51) Int. Cl. C12N 5/00; C12M 1/12; C12M 1/00

(52) U.S. Cl. CPC C12N 5/0062; C12M 25/14; C12N 2511/00; C12N 2513/00; C12N 2533/90; C12M 29/10

(57) ABSTRACT

Described herein are systems and methods relating to three-dimensional (3D) cell manufacturing utilizing a 3D growth media. According to aspects of the present disclosure, pluralities of cells and ECM components can be printed into a bioreactor as a sheet. Sheets can be retrieved, digested, split, and re-printed, thereby expanding the number of cells in an efficient manner that conserves space in a tissue culture incubator, conserves bioreactor consumables, and consumes nutrients required for cell maintenance and growth.

### CROSS-REFERENCE TO RELATED APPLICATION

This application claims priority to U.S. Provisional Application entitled “SYSTEMS AND METHODS RELATING TO THREE-DIMENSIONAL (3D) CELL MANUFACTURING,” having Ser. No. 62/869,308, filed on Jul. 1, 2019, which is entirely incorporated herein by reference.

### GOVERNMENT SUPPORT

This invention was made with government support under Grant No. DMR1352043 awarded by the National Science Foundation. The government has certain rights in the invention.

### BACKGROUND

Developing advanced techniques capable of mass-producing high-quality cells within the US will transform the healthcare industry, improve the health of millions of citizens, and expand the US economy by growing the emerging biomanufacturing industry.

Among many challenges to the development of such advanced techniques, a critical barrier preventing the establishment and growth of the biomanufacturing industry is the problem of scale—the major unmet need for high-volume production of qualified living cells to serve as building material. Manufactured tissue constructs for tissue engineering, regenerative medicine, and drug screening all require huge numbers of functionally homogeneous and well-characterized cells. A currently available mass production approach that comes closest to meeting bio-manufacturing needs is the “cell factory”—a large culture vessel in which cells grow on tissue culture plastic. While cell factories are designed for parallelization by stacking and inter-connecting them with liquid media exchange systems, they suffer from being inherently 2D systems; monolayers are so thin that 10 cell factories at maximum capacity only hold enough cells to create a piece of dense tissue the size of a grape (FIG. 1A). The typical laboratory incubator could accommodate about 30 cell factories with modifications. Thus, manufacturing thousands or tens of thousands of small tissues per day would correspondingly require thousands to tens of thousands of incubators, and all the related infrastructure and human support.

Even if this problem of scale was solved, it is now widely recognized that culturing cells adhered to 2D tissue culture plastic generally alters cell phenotype and can impact stem cell differentiation. Such alteration of phenotype and differentiation precludes the true utility of maximizing the benefits of in vitro culture systems. Failing to overcome the barriers associated with production scale and current 2D practices will absolutely stifle the biomanufacturing industry, coming at the cost of the health of millions of people by impeding the development of novel therapeutics. Accordingly, there is a need to address the aforementioned deficiencies and inadequacies.

### SUMMARY

Described herein are systems, methods, and kits relating to 3D cell culture. In embodiments according to the present disclosure, methods of 3D culture as described herein can comprise: printing a plurality of cells into a bioreactor comprising a three-dimensional (3D) culture medium with a printing device, wherein the 3D cell culture medium comprises a plurality of hydrogel particles and a liquid cell culture medium, wherein the hydrogel particles are swelled with the liquid cell culture medium to form a granular gel; incubating the plurality of printed cells in the bioreactor; re-collecting the incubated plurality of printed cells; digesting the collected plurality of printed cells; splitting the digested plurality of cells; and re-printing the diluted plurality of cells to expand populations.

In embodiments of methods according the present disclosure, the bioreactor can be a perfusion-enabled bioreactor.

In embodiments of methods according the present disclosure, the printing device can comprise three orthogonal translation axes.

In embodiments according to the present disclosure, methods as described herein further comprise printing one or more extra-cellular matrix (ECM) structures with the printing device in the bioreactor before incubating.

In embodiments of methods according the present disclosure, the one or more ECM structures can comprise collagen-1, human placental matrix or components thereof, Matrigel® (the registered trade name for a gelatinous protein mixture secreted by Engelbreth-Holm-Swarm mouse sarcoma cells), or laminin.

In embodiments of methods according the present disclosure, the one or more ECM structures can be printed in a sheet with a longest dimension extending from the bottom of the bioreactor to the top.

In embodiments of methods according the present disclosure, the plurality of cells can be mixed with 3D culture medium before printing.

In embodiments of methods according the present disclosure, the one or more ECM structures can be mixed with 3D culture medium before printing.

In embodiments of methods according the present disclosure, the 3D culture medium can have a yield stress such that the cell growth medium undergoes a phase change from a first solid phase to a second liquid phase upon application of a shear stress greater than the yield stress.

In embodiments of methods according the present disclosure, the yield stress can be on the order of 10 Pa.

In embodiments of methods according the present disclosure, the concentration of hydrogel particles can be between 0.05% to about 1.0% by weight.

In embodiments of methods according the present disclosure, the hydrogel particles can have a size between about 0.1 μm to about 100 μm when swollen with the liquid cell culture medium.

In embodiments of methods according the present disclosure, the 3D culture medium can have a pore size of about 25 nm to about 25 μm.

In embodiments of methods according the present disclosure, a plurality of cells can be disposed in a region of the 3D cell culture medium.

In embodiments of methods according the present disclosure, the incubating can be for a time period of about 12 hours to about 240 hours.

In embodiments of methods according the present disclosure, the incubating can be at a temperature of about 30° C. to about 40° C.

In embodiments of methods according the present disclosure, the incubating can be a pO₂ of greater than about 10 kPa

In embodiments of methods according the present disclosure, the re-collecting can be performed with a syringe needle having a bore diameter of about 0.05 mm to about 20 mm.

In embodiments of methods according the present disclosure, the syringe needle can be operably connected to the printing device.

In embodiments of methods according the present disclosure, the digesting can be an enzymatic digestion.

In embodiments of methods according the present disclosure, the enzymatic digestion comprises trypsin.

In embodiments of methods according the present disclosure, the splitting is by a splitting ratio of about 1:2 to about 1:20.

Described herein are systems for 3D cell manufacturing. In embodiments according to the present disclosure, systems for 3D cell manufacturing can comprise: a printing device; and 3D culture medium.

In embodiments according to the present disclosure, systems for 3D cell manufacturing can further comprise one or more bioreactors configured to receive 3D culture medium and a printed plurality of cells, ECM structures, or both.

In embodiments according to the present disclosure, systems for 3D cell manufacturing can further comprise a plurality of cells.

In embodiments according to the present disclosure, systems for 3D cell manufacturing can further comprise a computing device operably connected to the printing device.

In embodiments according to the present disclosure, systems for 3D cell manufacturing can further comprise ECM materials.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A-1C: The “cell factory”, a large culture dish designed with interconnected ports for stacking and perfusion, is a standard of practice in 2D cell manufacturing. Approximately 30 cell factories could be fit into a standard incubator with some modifications, holding enough cells to produce a dense tissue the size of a grape (FIG. 1A). By contrast, 3D printing extra cellular matrix (ECM) structures containing cells into a packed microgel 3D growth medium, just three 96-well plates could achieve the same cell throughput, given sufficient liquid perfusion (FIG. 1B). Using this new growth medium enables the full “cell culture loop” to be performed automatically in 3D without direct handling by a technician (FIG. 1C).

FIGS. 2A-2D illustrate systems and methods according to the present disclosure. 3D bioprinting methods are most often developed with the goal of creating tissue-mimicking structures for implantation or drug screening applications. Our recently developed 3D bioprinting method can support extremely fine and delicate structures made from cells and ECM, allowing its adaptation into a 3D cell culture technology compatible with automation (FIG. 2A). 3D printing and culture medium as described herein is made from packed microgels, creating a network of perfusable pore space (FIG. 2B). The pore-space permeability can be controlled by the microgel size (see FIGS. 2B and 6A). It is proposed to create large, thin structures, ensuring that all cells are within 100 μm of perfusing liquid media. The microgel medium can yield and flow under stresses exceeding 0.2-10 Pa, allowing the insertion of instruments to controllably retrieve cell-ECM structures for passaging, sampling, or stimulation (FIG. 2C). Attempting to 3D print cells into pre-existing ECM structures will uncontrollably damage, deform, or collapse the networks. Similarly, using popular “bioinks” like F-127 pluronic, in place of the microgel medium as described herein, creating crevasses; such bioinks are not perfusable (FIG. 2D).

FIGS. 3A-3C: Time-release polymer films were loaded with fluorescent dyes that are activated by cellular metabolism once in the cytosol of HUH7 hepatocytes (green hues, 5-chloromethylfluorescein diacetate, CMFDA; red hues, calcein red-orange AM; FIG. 3A). A 2D gradient of dye uptake develops in spheroids made from HUH7 cells, where green signal is dominant in the upper right, red signal is dominant in the lower left, an intense mix of red and green is observed in the upper left, and the weakest combined signal is seen in the lower right (FIG. 3B). To show that large 3D printed arrays are achievable, 1000 spheres made from fluorescent colloids were printed in 1 h, shown here in a 2 in. quartz cube (FIG. 3C).

FIGS. 4A-4C: The fabrication and stability of high-aspect ratio structures made from cells and ECM, printed into microgel medium (3t3 fibroblasts and collagen-1), has been systematically explored (FIG. 4A). By varying the properties of the microgel culture medium, it can be controlled whether long, thin cylinders buckle, break up, contract axially, or remain static. Extremely thin macro-scale sheets (3D rendering of confocal fluorescence data, FIG. 4B) have also been printed, which demonstrate the ability to perform the proposed passaging steps (red 3t3+green collagen-1; FIG. 4C).

FIGS. 5A-5D: Perfusion chamber inserts were designed to snap-and-seal into 12-well plates, compatible with the plate lid (FIGS. 5A-5B). Within each well, the central chamber is used to 3D print cell-ECM structures and supply liquid growth media. The outer chamber is used to collect waste media (FIG. 5C). Preliminary tests show that gravity provides a sufficient pressure gradient to drive fluids from the feeding chamber to the waste chamber (FIG. 5C). Should controlled, continuous flow be necessary, pressurized perfusion tubes can be attached and sealed to the ports of the insert. The surface roughness of the fabricated structure can be chosen to determine fluid permeability while preventing microgel motion, trapping the gels in the percolated pore space between the base of the insert and the bottom of the well-plate (FIG. 5D).

FIGS. 6A-6C: The permeability, K, of microgel growth media is found by measuring the flow of a column of liquid growth media driven by gravity though a pack of length, L. The pore size in different microgel systems having particles of different diameter are estimated (FIG. 6A). Perfusion of 3D printed spheroids within microgel growth medium eliminates necrosis for up to 28 days (MG63 cells, hematoxylin and eosin stain; FIG. 6B). Models can predict the combinations of flowrate, cell metabolic rate, and molecular diffusivity required to support cells at a given volume fraction (FIG. 6C).

FIG. 7 is a graft of human placental matrix (hPM) effect on cell proliferation. Endothelial colony forming cells (ECFCs), obtained from whole bone marrow density gradient separation, show the addition of hPM significantly increased proliferation without driving angiogenic differentiation.

FIG. 8 is a photomicrograph demonstrating current work on drug-induced liver injury in 3D printed liver models in vitro, although this figure is not focused on cell manufacturing. Hepatocyte toroids (green) in a sea of fibroblasts (red), are supported by the 3D microgel medium.

FIGS. 9A-9B demonstrate measurements of intracellular ATP with CellTiter-Glo® kits as an estimate of metabolic level. FIG. 9A: Calibration curves were collected in liquid media and three different microgel formulations. FIG. 9B: Within experimental variability, cells cultured in the three different microgel materials exhibited the same levels of intracellular ATP as that measured from cells grown in liquid media. These assays can be performed using the newly developed charge-neutral microgel system.

FIG. 10 is an embodiment of an apparatus 1010 which represents aspects of systems and methods of the present disclosure.

FIG. 11 is an embodiment of a method according to the present disclosure.

FIG. 12 is an additional embodiment of a method according to the present disclosure.

FIG. 13 is an embodiment of an extrusion (i.e. printing) system that can be employed according to systems and methods as disclosed herein.

FIG. 14 is another diagram of an embodiment of a computing system on which embodiments according to the present disclosure can be implemented.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of mechanical engineering, fluid motion, fluid dynamics, mechanical engineering, software engineering, cellular biology, tissue culture, and the like.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

About: The term “about”, when used herein in reference to a value, refers to a value that is similar, in context to the referenced value. In general, those skilled in the art, familiar with the context, will appreciate the relevant degree of variance encompassed by “about” in that context. In certain aspects, “about” is ±5% of the reference value.

Associated with: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition”, as used herein, can be used to refer to a discrete physical entity that comprises one or more specified components. In general, unless otherwise specified, a composition can be of any form—e.g., gas, gel, liquid, solid, etc.

Comprising: A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

“Improved,” “increased” or “reduced”: As used herein, these terms, or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Sample: As used herein “sample” refers to a plurality of cells that can be 3D printed according to aspects of the present disclosure. In certain aspects, a sample can further comprise one or more extracellular matrix (ECM) components). In additional aspects, a sample can further comprise 3D cell culture media as described herein.

Sheet: As used herein, “sheet” refers to a composition comprising a plurality of printed cells into a three-dimensional structure having a width less than the length or height. In aspects according to the present disclosure, a sheet can further comprise one or more extracellular components and/or structures, extracellular matrix components especially.

### DISCUSSION

Cell Manufacturing.

Despite the barriers to industrial-scale manufacturing describe above, several methods are effective for producing large clinical-scale populations of adherent cells. For example, extremely large culture-plates, or “cell factories”, have been used to produce cell populations large enough for clinical trials, including bone marrow stromal cells, mesenchymal stem cells (MSCs) for multiple sclerosis therapies, among other efforts to demonstrate clinically-relevant MSC expansion. The demands associated with culture space and manual labor, however, make these approaches excessively costly for industrial-scale production. One promising method that may overcome these limitations is the Quantum Cell Expansion System (QCES), which grows cells on the inner surfaces of hollow fibers, bundled into cartridges that connect to fluidic control systems. The fibers are made from polystyrene treated to promote cell attachment. While this system shows promise, the cell culture environment is essentially the same as that of traditional 2D tissue culture plastic.

Another potentially space-saving approach is the packed bed bioreactor, in which mm-scale pellets to which cells adhere, most often made from polystyrene, are packed into a perfusable container. Like the QCES, packed bed bioreactors leverage 3D to pack cells into a small space, while the cells feel an essentially 2D plastic microenvironment. Packed bed bioreactors, cell factories, and the QCES all rely on perfusion to exchange liquid media. Another way to expand adherent cell populations is to use “microcarriers” in stirred bioreactors. Microcarriers are beads on which attached cells will proliferate, most commonly made from dextran, glass, gelatin, polystyrene, or cellulose, often with surface modification. The shear stress on cells from hydrodynamic forces and the difficulty of separating cells from microcarriers without cell damage remains a major concern. Microencapsulation of cells inside of hydrogel microcarriers presents one potential solution to the problems of hydrodynamic stresses, but the material costs and the challenges of encapsulation, decapsulation, and cell separation have limited their use. In contrast to all the other approaches to cell production, however, microencapsulation is the only approach that provides cells with a 3D ECM microenvironment, which is believed to be critical for quality cell production, moving forward.

Microenvironment: 2D vs. 3D

The dramatic differences between cells grown on 2D surfaces and cells in vivo or in 3D culture are known. Cell shape, structure, motion, mechanical behavior, and chromosome conformation in 3D are different from those in the dish, and cells grown in monolayers exhibit gene expression profiles that do not correlate or are anticorrelated with those of cells grown in 3D culture or xenograft animal models. It is therefore becoming widely recognized that 3D microenvironments drive cells toward in vivo phenotypes to a far greater extent than 2D culture. For example, 3D cultures of human mammary epithelial cells form acinar structures similar to breast tissue with characteristic lumens. Furthermore, these acini are functional in the sense that they secrete proteins characteristic of in vivo tissue. 3D culture has been used to promote differentiation of human pluripotent cells into neuronal lineages producing dopamine. While this evidence strongly suggests that 3D microenvironments are critical to growing cells with the correct phenotype and gene expression patterns, current understanding of cell biology has been heavily shaped by 2D cell culture methods. In cellular mechanobiology, extensive study of migration, morphology, cytoskeletal architecture, and mechanical activity on flat surfaces has provided a robust framework for describing cell behavior. In molecular cell biology, immunofluorescence assays combined with well-plates and plate-readers have streamlined the characterization of cells by protein production levels. Closing this major gap between 2D in vitro culture practices and in vivo biology requires a tunable and flexible method for culturing cells in 3D ECM microenvironments; to enable the growth of the biomanufacturing industry, this approach must be scalable to expand cells at industrial levels.

3D Scaffolds and Gels for Cell Culture

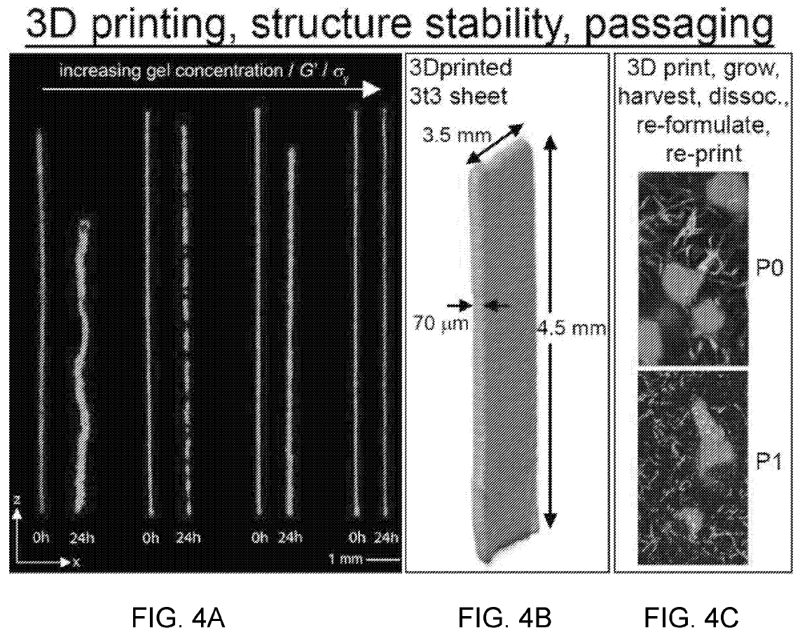

Cells thrive in natural 3D biopolymer networks like collagen and Matrigel® (trade name for a gelatinous protein mixture secreted by Engelbreth-Holm-Swarm mouse sarcoma cells), as well as in engineered materials with motifs for adhesion and enzymatic degradation that facilitate cell growth and migration. The mechanical properties of these matrices prevent the precise placement or structuring of cells in 3D space, and impede continual cell culture that uses serial passaging, harvesting, and direct assaying through fluid exchange; branched or crosslinked biopolymer networks are irreversibly damaged when yielded by the insertion of instruments and entangled polymer networks flow over long time-scales. These drawbacks are well-recognized and have led to the development of adaptable hydrogel networks with specially designed linkages that can be reversed. Numerous different chemical and physical approaches have been developed, but the unifying concept is that the chemical equilibrium between linked and unlinked moieties kinetically controls the breaking and reforming of network junctions under localized stresses or other stimuli. 3D printing of hydrogels and cells into such adaptable networks has been demonstrated, yet these gels exhibit fluid like rheology at low frequencies, creating new variables that must be harnessed if used as a stabilizing medium for culturing cells in 3D ECM structures. While other 3D bioprinting methods have been extremely effective at producing functional and implantable structures, the materials and approaches in these tissue-biofabrication efforts are not designed for the mass production of cells. In contrast to these approaches for supporting cells in 3D, recent development of a combined 3D printing and 3D culture medium made from packed microgels can enable all the criteria for 3D cell production to be met: natural ECM containing cells can be printed into structures at high-resolution (see FIG. 4A, for example); the packed microgels exhibit solid-like rheological properties at timescales exceeding 104 seconds and structures are observed to be stable for more than a week; expanding cells can be harvested, separated, and re-printed.

Perfusion Through 3D Culture Environments

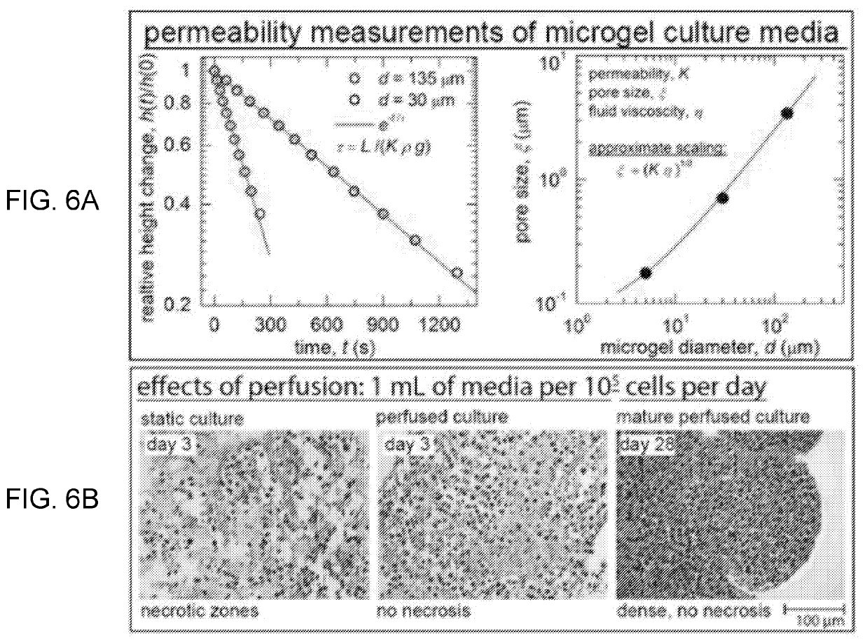

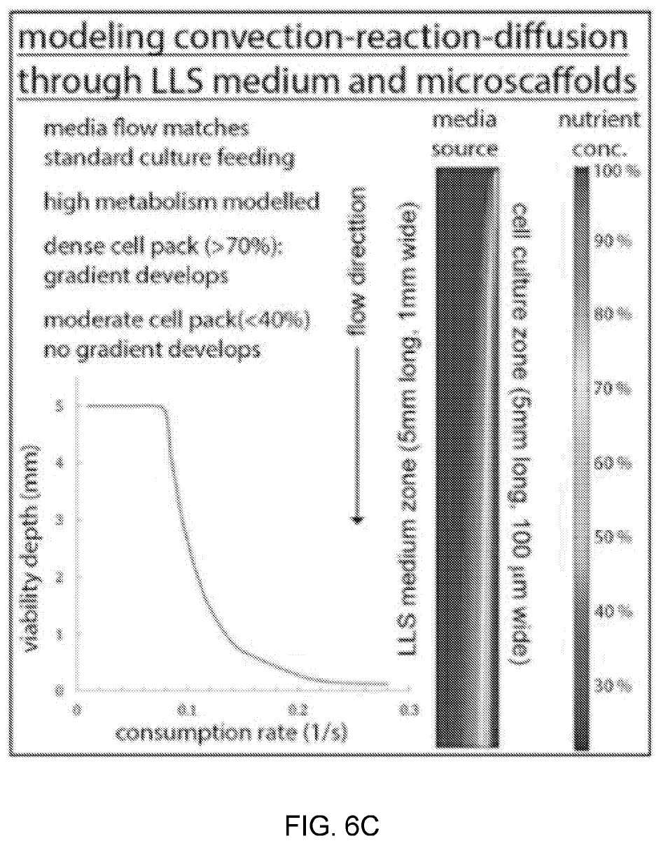

Culturing large cell populations in small spaces requires liquid perfusion to provide O₂ and nutrients to cells and remove waste. Perfusing liquid through homogeneous hydrogels is challenging; crosslinked hydrogels require large pressure gradients to induce uniform flows over large volumes because of their low hydraulic permeability. Combined with their soft mechanical properties, large deformations and fracture can occur at the flow rates needed for uniform perfusion of cells. Structured and reinforced hydrogels have been developed to improve perfusion characteristics. Hydrogels with embedded carbon foam have been used to improve mechanical stability. An alternative approach is to increase hydraulic permeability by increasing pore space or pore connectivity, sometimes employing foaming agents. Preferential flow routes can be introduced, for example by molded microchannels, providing low resistance pathways to facilitate widespread perfusion. Without modification, microgels have suitable perfusion properties because of large pore spaces between gel particles at low packing densities; a diversity of perfusable scaffolds made from functionalized and crosslinked microgels have been developed. In studies as described herein, when cell-ECM structures are 3D printed, surrounding microgels provide low resistance regions through which nutrients can be supplied and wastes removed by perfusion. This approach is promising since advection allows for nutrient coverage over larger volumes and more uniformly than by diffusion alone. In studies as described herein stability of cell-ECM structures and packed microgels at high perfusion rates has been shown (see, for example, FIGS. 6A-6C).

Described herein are embodiments of automated 3D cell culture technology and related methods for large-scale production of high-quality cells. By shifting current practices to automated 3D culture, the problems and scale and cell quality can simultaneously be addressed and solved, and methods of 2D culture can be improved upon; larger numbers of cells arranged in 3D distributions can be grown in dramatically smaller perfusion bioreactors; the extracellular matrix (ECM) that can host 3D cell populations can eliminate the deleterious effects of tissue culture plastic on cell phenotype.

As described herein, recent advances in biofabrication technology are transformed into cell manufacturing tools. Specifically, the unique properties of packed microgel 3D printing media can be leveraged to design cell ECM structures for large-scale cell production. The 3D growth medium made from packed microgels swollen in liquid culture media can enable the “cell culture loop” to be performed automatically, in which 3D cell-ECM structures are printed, incubated, re-collected, digested, diluted, and re-printed to expand populations.

As described herein, the pore-space in microgel packs can also be leveraged to exchange nutrients and waste with large 3D cell populations through perfusion: an important factor related to cell viability and metabolism through diffusive transport in the 3D-cell-ECM structures is controlling the 3D flow distribution of liquid media through porous microgels. The well-defined 3D culture conditions control cell phenotype, gene expression, and stem cell differentiation in 3D; a combination of media formulation, flow control, and ECM composition can enable the production of high-quality cells with the potential for industrial-scale up. Systems and methods according to the present disclosure can help grow the biomanufacturing industry within the US and improve the health of the American populace.

3D Culture Medium

Liquid-like solid (LLS) three-dimensional (3D) cell growth medium (also referred to herein as “liquid-like solid”, “LLS”, “3D growth medium”, “3D cell growth medium”, “3D culture medium” or “granular microgel”) for use in with the disclosed systems and methods described herein is disclosed in WO2016182969A1 by Sawyer et al., which is incorporated by reference in its entirety for the description of how to make and use this LLS medium.

Liquid-like solids (LLS) have properties that provide a combination of transport, elastic, and yielding properties, which can be leveraged to design a support material for the maintenance of living cells in three-dimensional culture. These materials may be composed predominantly of solvent that freely diffuses and can occupy more than 99% of their volume, but they also have a finite modulus and extremely low yield-stress in their solid state. Upon yielding, these materials shear and behave like classical fluids. Packed granular microgels are a class of liquid-like solids that have recently been adopted as a robust medium for precise three dimensional fabrication of delicate materials. The unrestricted diffusion of nutrients, small molecules, and proteins can support the metabolic needs of cells and provide an easy route to the development of combinatorial screening methods. Unperturbed, LLS materials can provide support and stability to cells and to cell-assemblies, and facilitate the development and maintenance of precise multi-cellular structures.

Briefly, the 3D cell growth medium may comprise hydrogel particles dispersed in a liquid cell growth medium. Any suitable liquid cell growth medium may be used; a particular liquid cell growth medium may be chosen depending on the types of cells which are to be placed within the 3D cell growth medium, as one of skill in the art would understand. For example, suitable cell growth medium may be human cell growth medium, murine cell growth medium, bovine cell growth medium or any other suitable cell growth medium. Depending on the particular embodiment, hydrogel particles and liquid cell growth medium may be combined in any suitable combination. For example, in some embodiments, a 3D cell growth medium comprises approximately 0.5% to 1% hydrogel particles by weight. In some embodiments, the hydrogel particles can have a size in the range of about 0.1 μm to about 100 μm when swollen with the liquid cell culture medium. In some embodiments, the hydrogel particles can have a size in the range of about 1 μm to about 10 μm when swollen with the liquid cell culture medium.

In accordance with some embodiments, the hydrogel particles may be made from a bio-compatible polymer.

The hydrogel particles may swell with the liquid growth medium to form a granular gel material. Depending on the particular embodiment, the swollen hydrogel particles may have a characteristic size at the micron or submicron scales. For example, in some embodiments, the swollen hydrogel particles may have a size between about 0.1 μm and 100 μm. Furthermore, a 3D cell growth medium may have any suitable combination of mechanical properties, and in some embodiments, the mechanical properties may be tuned via the relative concentration of hydrogel particles and liquid cell growth medium. For example, a higher concentration of hydrogel particles may result in a 3D growth medium having a higher elastic modulus and/or a higher yield stress.

According to some embodiments, the 3D cell growth medium may be made from materials such that the granular gel material undergoes a temporary phase change due to an applied stress (e.g. a thixotropic or “yield stress” material). Such materials may be solids or in some other phase in which they retain their shape under applied stresses at levels below their yield stress. At applied stresses exceeding the yield stress, these materials may become fluids or in some other more malleable phase in which they may alter their shape. When the applied stress is removed, yield stress materials may become solid again. Stress may be applied to such materials in any suitable way. For example, energy may be added to such materials to create a phase change. The energy may be in any suitable form, including mechanical, electrical, radiant, or photonic, etc.

Regardless of how cells are placed in the medium, the yield stress of the yield stress material may be large enough to prevent yielding due to gravitational and/or diffusional forces exerted by the cells such that the position of the cells within the 3D growth medium may remain substantially constant over time. As described in more detail below, placement and/or retrieval of groups of cells may be done manually or automatically.

A yield stress material as described herein may have any suitable mechanical properties. For example, in some embodiments, a yield stress material may have an elastic modulus between approximately 1 Pa and 1000 Pa when in a solid phase or other phase in which the material retains its shape under applied stresses at levels below the yield stress. In some embodiments, the yield stress required to transform a yield stress material to a fluid-like phase may be between approximately 1 Pa and 1000 Pa. In some embodiments, the yield stress may be on the order of 10 Pa, such as 10 Pa+/−25%. When transformed to a fluid-like phase, a yield stress material may have a viscosity between approximately 1 Pa s and 10,000 Pa s. However, it should be understood that other values for the elastic modulus, yield stress, and/or viscosity of a yield stress material are also possible, as the present disclosure is not so limited.

A group of cells may be placed in a 3D growth medium made from a yield stress material via any suitable method. For example, in some embodiments, cells may be injected or otherwise placed at a particular location within the 3D growth medium with a syringe, pipette, or other suitable placement or injection device, such as automated liquid handler. In some embodiments an array of automated cell dispensers may be used to inject multiple cell samples into a container of 3-D growth medium. Movement of the tip of a placement device through the 3D growth medium may impart a sufficient amount of energy into a region around the tip to cause yielding such that the placement tool may be easily moved to any location within the 3D growth medium. In some instances, a pressure applied by a placement tool to deposit a group of cells within the 3D growth medium may also be sufficient to cause yielding such that the 3D growth medium flows to accommodate the group of cells. Movement of a placement tool may be performed manually (e.g. “by hand”), or may performed by a machine or any other suitable mechanism.

In some embodiments, multiple independent groups of cells may be placed within a single volume of a 3D cell growth medium. For example, a volume of 3D cell growth medium may be large enough to accommodate at least 2, at least 5, at least 10, at least 20, at least 50, at least 100, at least 1000, or any other suitable number of independent groups of cells. Alternatively, a volume of 3D cell growth medium may only have one group of cells. Furthermore, it should be understood that a group of cells may comprise any suitable number of cells, and that the cells may of one or more different types.

Depending on the particular embodiment, groups of cells may be placed within a 3D cell growth medium according to any suitable shape, geometry, and/or pattern. For example, independent groups of cells may be deposited as spheroids, and the spheroids may be arranged on a 3D grid, or any other suitable 3D pattern. The independent spheroids may all comprise approximately the same number of cells and be approximately the same size, or alternatively different spheroids may have different numbers of cells and different sizes. In some embodiments, cells may be arranged in shapes such as embryoid or organoid bodies, tubes, cylinders, toroids, hierarchically branched vessel networks, high aspect ratio objects, thin closed shells, or other complex shapes which may correspond to geometries of tissues, vessels or other biological structures.

According to some embodiments, a 3D cell growth medium made from a yield stress material may enable 3D printing of cells to form a desired pattern in three dimensions. For example, a computer-controlled injector tip may trace out a spatial path within a 3D cell growth medium and inject cells at locations along the path to form a desired 3D pattern or shape. Movement of the injector tip through the 3D cell growth medium may impart sufficient mechanical energy to cause yielding in a region around the injector tip to allow the injector tip to easily move through the 3D cell growth medium, and also to accommodate injection of cells. After injection, the 3D cell growth medium may transform back into a solid-like phase to support the printed cells and maintain the printed geometry. However, it should be understood that 3D printing techniques are not required to use a 3D growth medium as described herein.

According to some embodiments, a 3D cell growth medium may be prepared by dispersing hydrogel particles in a liquid cell growth medium. The hydrogel particles may be mixed with the liquid cell growth medium using a centrifugal mixer, a shaker, or any other suitable mixing device. During mixing, the hydrogel particles may swell with the liquid cell growth medium to form a material which is substantially solid when an applied shear stress is below a yield stress, as discussed above. After mixing, entrained air or gas bubbles introduced during the mixing process may be removed via centrifugation, agitation, or any other suitable method to remove bubbles from 3D cell growth medium.

In some embodiments, preparation of a 3D cell growth medium may also involve buffering to adjust the pH of a hydrogel particle and liquid cell growth medium mixture to a desired value. For example, some hydrogel particles may be made from polymers having a predominantly negative charge which may cause a cell growth medium to be overly acidic (have a pH which is below a desired value). The pH of the cell growth medium may be adjusted by adding a strong base to neutralize the acid and raise the pH to reach the desired value. Alternatively, a mixture may have a pH that is higher than a desired value; the pH of such a mixture may be lowered by adding a strong acid. According to some embodiments, the desired pH value may be in the range of about 7.0 to 7.4, or, in some embodiments 7.2 to 7.6, or any other suitable pH value which may, or may not, correspond to in vivo conditions. The pH value, for example may be approximately 7.4. In some embodiments, the pH may be adjusted once the dissolved CO₂ levels are adjusted to a desired value, such as approximately 5%.

Yield stress can be measured by performing a strain rate sweep in which the stress is measured at many constant strain rates. Yield stress can be determined by fitting these data to a classic Herschel-Bulkley model (σ=σ_{y}+k\dot{γ}ⁿ). (b) To determine the elastic and viscous moduli of non-yielded LLS media, frequency sweeps at 1% strain can be performed. The elastic and viscous moduli remain flat and separated over a wide range of frequency, behaving like a Kelvin-Voigt linear solid with damping. Together, these rheological properties demonstrate that a smooth transition between solid and liquid phases occurs with granular microgels, facilitating their use as a 3D support matrix for cell printing, culturing, and assaying.

An example of a hydrogel with which some embodiments may operate is a carbomer polymer, such as Carbopol®. Carbomer polymers may be polyelectrolytic, and may comprise deformable microgel particles. Carbomer polymers are particulate, high-molecular-weight crosslinked polymers of acrylic acid with molecular weights of up to 3-4 billion Daltons. Carbomer polymers may also comprise co-polymers of acrylic acid and other aqueous monomers and polymers such as poly-ethylene-glycol.

While acrylic acid is a common primary monomer used to form polyacrylic acid the term is not limited thereto but includes generally all α-β unsaturated monomers with carboxylic pendant groups or anhydrides of dicarboxylic acids and processing aids as described in U.S. Pat. No. 5,349,030. Other useful carboxyl containing polymers are described in U.S. Pat. No. 3,940,351, directed to polymers of unsaturated carboxylic acid and at least one alkyl acrylic or methacrylic ester where the alkyl group contains 10 to 30 carbon atoms, and U.S. Pat. Nos. 5,034,486; 5,034,487; and 5,034,488; which are directed to maleic anhydride copolymers with vinyl ethers. Other types of such copolymers are described in U.S. Pat. No. 4,062,817 wherein the polymers described in U.S. Pat. No. 3,940,351 contain additionally another alkyl acrylic or methacrylic ester and the alkyl groups contain 1 to 8 carbon atoms. Carboxylic polymers and copolymers such as those of acrylic acid and methacrylic acid also may be cross-linked with polyfunctional materials as divinyl benzene, unsaturated diesters and the like, as is disclosed in U.S. Pat. Nos. 2,340,110; 2,340,111; and 2,533,635. The disclosures of all of these U.S. patents are hereby incorporated herein by reference for their discussion of carboxylic polymers and copolymers that, when used in polyacrylic acids, form yield stress materials as otherwise disclosed herein. Specific types of cross-linked polyacrylic acids include carbomer homopolymer, carbomer copolymer and carbomer interpolymer monographs in the U.S. Pharmocopia 23 NR 18, and Carbomer and C10-30 alkylacrylate crosspolymer, acrylates crosspolymers as described in PCPC International Cosmetic Ingredient Dictionary & Handbook, 12th Edition (2008).

Carbomer polymer dispersions are acidic with a pH of approximately 3. When neutralized to a pH of 6-10, the particles swell dramatically. The addition of salts to swelled Carbomer can reduce the particle size and strongly influence their rheological properties. Swelled Carbomers are nearly refractive index matched to solvents like water and ethanol, making them optically clear. The original synthetic powdered Carbomer was trademarked as Carbopol® and commercialized in 1958 by BF Goodrich (now known as Lubrizol), though Carbomers are commercially available in a multitude of different formulations.

Hydrogels may include packed microgels—microscopic gel particles, ˜5 μm in diameter, made from crosslinked polymer. The yield stress of Carbopol® is controlled by water content. Carbopol® yield stress can be varied between about 1 Pa to about 1000 Pa. Thus, both materials can be tuned to span the stress levels that cells typically generate. As discussed above, while materials may have yield stresses in a range of 1-1000 Pa, in some embodiments it may be advantageous to use yield stress materials having yield stresses in a range of 1-100 Pa or 10-100 Pa. In addition, some such materials may have thixotropic times less than 2.5, less than 1.5 seconds, less than 1 second, or less than 0.5 seconds, and greater than 0.25 seconds or greater than 0.1 seconds, and/or thixotropic indexes less than 7, less than 6.5, or less than 5, and greater than 4, or greater than 2, or greater than 1.

In one non-limiting example, a 3D cell growth medium comprises approximately 0.2% to about 0.7% by mass Carbopol® particles (Lubrizol). The Carbopol® particles are mixed with and swell with any suitable liquid cell growth medium, as described above, to form a 3D cell growth medium which comprises approximately 99.3% to about 99.8% by mass cell growth medium. After swelling, the particles have a characteristic size of about 1 μm to about 10 μm. The pH of the mixture is adjusted to a value of about 7.4 by adding a strong base, such as NaOH. The resulting 3D cell growth medium is a solid with a modulus of approximately 100-300 Pa, and a yield stress of approximately 20 Pa. When a stress is applied to this 3D cell growth medium which exceeds this yield stress, the cell growth medium transforms to a liquid-like phase with a viscosity of approximately 1 Pa s to about 1000 Pa s. As described above, the specific mechanical properties may be adjusted or tuned by varying the concentration of Carbopol®. For example, 3D cell growth media with higher concentrations of Carbopol® may be stiffer and/or have a larger yield stress.

In an embodiment, a LLS can be prepared with 0.9% (w/v) Carbopol® ETD 2020 polymer (Lubrizol Co.) was dispersed in cell growth media under sterile conditions. The pH of the medium is adjusted by adding NaOH until pH 7.4 is reached under the incubation condition of 37° C. and 5% CO₂, and the completely formulated material is homogenized in a high-speed centrifugal mixer. Carbopol® ETD 2020 swells maximally at this pH, making it suitable for cell culture applications. The gel medium was incubated at 37° C. and 5% CO₂.

The hydrogels for the LLS may be dispersed in solutions (e.g., solutions with cell growth medium) in various concentrations to form the LLS. One example of a concentration is below 2% by weight. Another concentration example is approximately 0.5% to 1% hydrogel particles by weight, and another is approximately 0.2% to about 0.7% by mass.

Hydrogels may include packed microgels—microscopic gel particles, ˜5 μL in diameter, made from crosslinked polymer. The yield stress of Carbopol® is controlled by water content. Carbopol® yield stress can be varied between roughly 1 and 1000 Pa. Thus, both materials can be tuned to span the stress levels that cells typically generate. As discussed above, while materials may have yield stresses in a range of 1-1000 Pa, in some embodiments it may be advantageous to use yield stress materials having yield stresses in a range of 1-100 Pa or 10-100 Pa. In addition, some such materials may have thixotropic times less than 2.5, less than 1.5 seconds, less than 1 second, or less than 0.5 seconds, and greater than 0.25 seconds or greater than 0.1 seconds, and/or thixotropic indexes less than 7, less than 6.5, or less than 5, and greater than 4, or greater than 2, or greater than 1.

Those skilled in the art will appreciate that materials having a yield stress will have certain thixotropic properties, such as a thixotropic time and a thixotropic index. As used herein, a thixotropic time is a time for shear stress to plateau following removal of a source of shear. The inventors recognize that thixotropic time may be measured in different ways. As used herein, unless indicated otherwise, thixotropic time is determined by applying to a material, for several seconds, a stress equal to 10 times the yield stress of the material, followed by dropping the stress to 0.1 times the yield stress. The amount of time for the shear rate to plateau following dropping of the stress is the thixotropic time.

As used herein, a thixotropic index (for a yield stress material) is defined as the ratio of viscosity at a strain-rate of 2 s¹ to viscosity at a strain-rate of 20 s¹.

Yield stress materials with desirable yield stresses may also have desirable thixotropic properties, such as desirable thixotropic indexes or thixotropic times. For example, desirable yield stress materials (including hydrogel materials having a yield stress below 100 Pascals, some of which are described in detail below, such as Carbopol® materials) may have thixotropic times less than 2.5 seconds, less than 1.5 seconds, less than 1 second, or less than 0.5 seconds, and greater than 0.25 seconds or greater than 0.1 seconds. An exemplary Carbopol® solution may exhibit a yield stress below 100 Pascals (and below 25 Pascals in some embodiments), as well as low thixotropic times. The thixotropic times of the Carbopol® solutions having a yield stress below 100 Pascals may be less than 2.5 seconds, less than 1.5 seconds, less than 1 second, or less than 0.5 seconds, and greater than 0.25 seconds or greater than 0.1 seconds.

In some embodiments, for hydrogel yield stress materials with a yield stress below 100 Pascals (including those discussed in detail below, like Carbopol® solutions), the thixotropic index is less than 7, less than 6.5, or less than 5, and greater than 4, or greater than 2, or greater than 1.

Desirable yield stress materials, like hydrogels such as the Carbopol® solutions described herein, may thus have thixotropic times less than 2.5, less than 1.5 seconds, less than 1 second, or less than 0.5 seconds, and greater than 0.25 seconds or greater than 0.1 seconds, and/or thixotropic indexes less than 7, less than 6.5, or less than 5, and greater than 4, or greater than 2, or greater than 1.

Because of the yield stress behavior of yield stress materials, materials deposited into a yield stress material (such as through 3D printing techniques described herein) may remain fixed in place in the yield stress material, without the yield stress material or the deposited material needing to be cured or otherwise treated to reverse a phase change (e.g., by heating to cross-link, following printing). Rather, the yield stress materials permit an indefinite working time on deposition of materials inside yield stress materials, including printing of cell clusters within yield stress materials.

In another non-limiting embodiment, a method for preparing a 3D cell growth medium is described. The method begins when hydrogel particles are mixed with a liquid cell culture medium. Mixing may be performed with a mechanical mixer, such as a centrifugal mixer, a shaker, or any other suitable mixing device to aid in dispersing the hydrogel particles in the liquid cell culture medium. During mixing, the hydrogel particles may swell with the liquid cell culture medium to form a granular gel, as discussed above. In some instances, the mixing act may result in the introduction of air bubbles or other gas bubbles which may become entrained in the gel. Such entrained gas bubbles are removed at via centrifugation, gentle agitation, or any other suitable technique. The pH of the mixture may then be adjusted; a base may be added to raise the pH, or alternatively an acid may be added to lower the pH, such until the pH of the mixture reaches a desired value. In some embodiments, the final pH value after adjustment is about 7.4.

Other aspects of 3D growth media described herein include embodiments such as those disclosed in U.S. Patent Publication Number 2018/0273743 A1 entitled “Crosslinkable or Functionalizable Polymers for 3D Printing of Soft Materials and filed on Jun. 1, 2018, which is incorporated by reference as if entirely set forth herein

Cells

Cells as described herein can be mammalian cells, in particular human, mouse, or rat cells. Cells as described herein can be stem cells or can be cells derived from mesoderm, endoderm, or ectoderm.

In certain embodiments, cells as described herein can be human perinatal stem cells. In certain embodiments, cells as described herein are induced-pluripotent stem cells. In certain embodiments, cells as described herein are fibroblasts. In certain embodiments, cells as described herein are mesenchymal stem cells (MSCs). As the skilled artisan would understand, cells as described herein are not limited to only the embodiments as described herein, and other cells can be suitable for use in systems and methods as described herein.

Additional examples of cells that can be printed utilizing systems and methods as described herein are provided in the Examples below.

Printing Systems

Printing systems as described herein can be a printing system with three orthongonal translation axes (x, y, z) that can moves a deposition needle or other disposition device around. Material deposition can be driven by volume displacement (e.g. with a syringe pump) or with controlled pressure from another pressure source, for example a vacuum. In embodiments, printing systems as described herein can further comprise one or more deposition needles. Printing systems as described herein can further be configured in an array, for example a multi-channel or full-plate array (8 channel, 24 channel, 96 channel, 384, and the like).

Printing systems can include cartridges or printers such as those disclosed in U.S. Patent Publication Number 2017/0361534 entitled “3D Printing Using Phase Changing Materials as Support” having a § 371 national stage entry date of Jun. 5, 2017, which is incorporated by reference as if entirely set forth herein.

Additional aspects of printing systems as described herein include apparati such as those disclosed in U.S. Patent Publication Number 2018/0258382 A1 entitled “Apparatus for Culturing and Interacting with a Three-Dimensional Cell Culture” having a § 371 national stage entry date of Mar. 13, 2018, which is incorporated by reference as if entirely set forth herein.

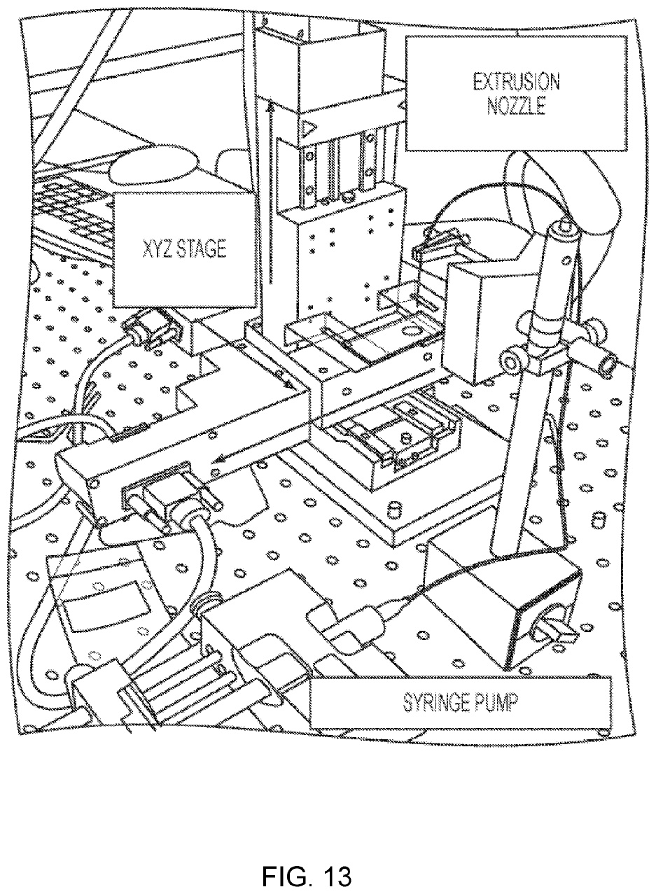

FIG. 13 depicts aspects of an embodiment of a printing system according to the present disclosure. In short, the 3D extrusion system may comprise an XYZ stage constructed from three linear translation stages (M-403, Physik Instrumente) driven by Mercury DC motor controllers (C-863, Physik Instrumente). The extrusion system is a computer-controlled syringe pump (Next Advance), held stationary to enable imaging as the stage moves, translating the yield stress support material in 3D (FIG. 13). The extrusion nozzles may be home made from glass pipettes, pulled with a Kopf-750 micropipette puller and shaped with a Narishige micro-forge. The inventors have control over nozzle diameter and shape; nozzle wettability is varied with hydrophilic 3-am inopropyl-triethoxysilane, or hydrophobic octadecyltriethoxysiloxane.

Computing Systems and Environments

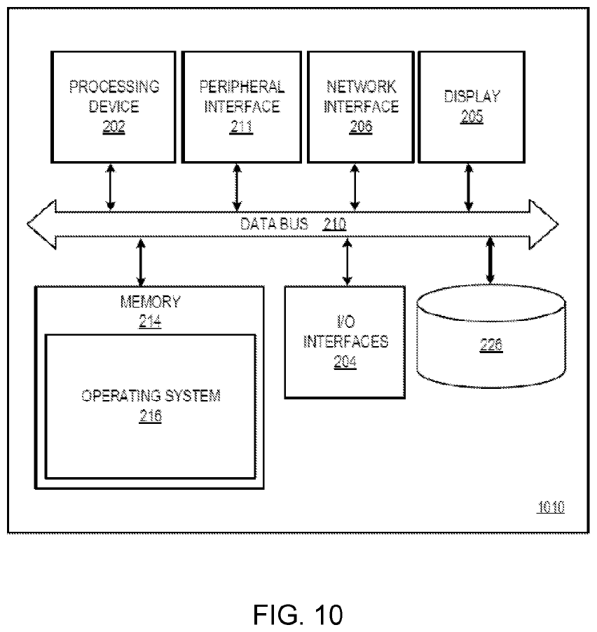

FIG. 10 depicts an apparatus 1010 in which the systems, printing devices, or other systems described herein may be coupled to in order to assist in automation of the system. Computing devices, such as depicted as apparatus 1010, can be coupled to a printing device as described herein so that data can be transferred between the printing system and the computing device. Computing devices can control aspects of the printing device, for example, movement of any one or more of the orthogonal translation axes or material deposition by sending data to the printing device. Such data can include instructions from a user of the device[s] as described herein.

The apparatus 1010 can be embodied in any one of a wide variety of wired and/or wireless computing devices, multiprocessor computing device, and so forth. As shown in FIG. 10, the apparatus 1010 comprises memory 214, a processing device 202, a number of input/output interfaces 204, a network interface 206, a display 205, a peripheral interface 211, and mass storage 226, wherein each of these devices are connected across a local data bus 210. The apparatus 1010 can be coupled to one or more peripheral measurement devices (not shown) connected to the apparatus 1010 via the peripheral interface 211.

The processing device 202 can include any custom made or commercially available processor, a central processing unit (CPU) or an auxiliary processor among several processors associated with the apparatus 1010, a semiconductor based microprocessor (in the form of a microchip), a macroprocessor, one or more application specific integrated circuits (ASICs), a plurality of suitably configured digital logic gates, and other well-known electrical configurations comprising discrete elements both individually and in various combinations to coordinate the overall operation of the computing system.

The memory 214 can include any one of a combination of volatile memory elements (e.g., random-access memory (RAM, such as DRAM, and SRAM, etc.)) and nonvolatile memory elements (e.g., ROM, hard drive, tape, CDROM, etc.). The memory 214 typically comprises a native operating system 216, one or more native applications, emulation systems, or emulated applications for any of a variety of operating systems and/or emulated hardware platforms, emulated operating systems, etc. For example, the applications can include application specific software which may be configured to perform some or all of the methods described herein (Labview, for example). In accordance with such embodiments, the application specific software is stored in memory 214 and executed by the processing device 202. One of ordinary skill in the art will appreciate that the memory 214 can, and typically will, comprise other components which have been omitted for purposes of brevity.

Input/output interfaces 204 provide any number of interfaces for the input and output of data. For example, where the apparatus 1010 comprises a personal computer, these components may interface with one or more user input devices 204. The display 205 can comprise a computer monitor, a plasma screen for a PC, a liquid crystal display (LCD) on a hand held device, or other display device.

In the context of this disclosure, a non-transitory computer-readable medium stores programs for use by or in connection with an instruction execution system, apparatus, or device. More specific examples of a computer-readable medium can include by way of example and without limitation: a portable computer diskette, a random access memory (RAM), a read-only memory (ROM), an erasable programmable read-only memory (EPROM, EEPROM, or Flash memory), and a portable compact disc read-only memory (CDROM) (optical).

With further reference to FIG. 10, network interface device 206 comprises various components used to transmit and/or receive data over a network environment. For example, the network interface 206 can include a device that can communicate with both inputs and outputs, for instance, a modulator/demodulator (e.g., a modem), wireless (e.g., radio frequency (RF)) transceiver, a telephonic interface, a bridge, a router, network card, etc.). The apparatus 1010 can communicate with one or more computing devices via the network interface 206 over a network. The apparatus 1010 may further comprise mass storage 226. The peripheral 211 interface supports various interfaces including, but not limited to IEEE-1394 High Performance Serial Bus (Firewire), USB, thunderbolt, a serial connection, and a parallel connection.

The apparatus 1010 shown in FIG. 10 can be embodied, for example, as a computing device coupled to a printing device. Printing devices can be a printing device as described herein.

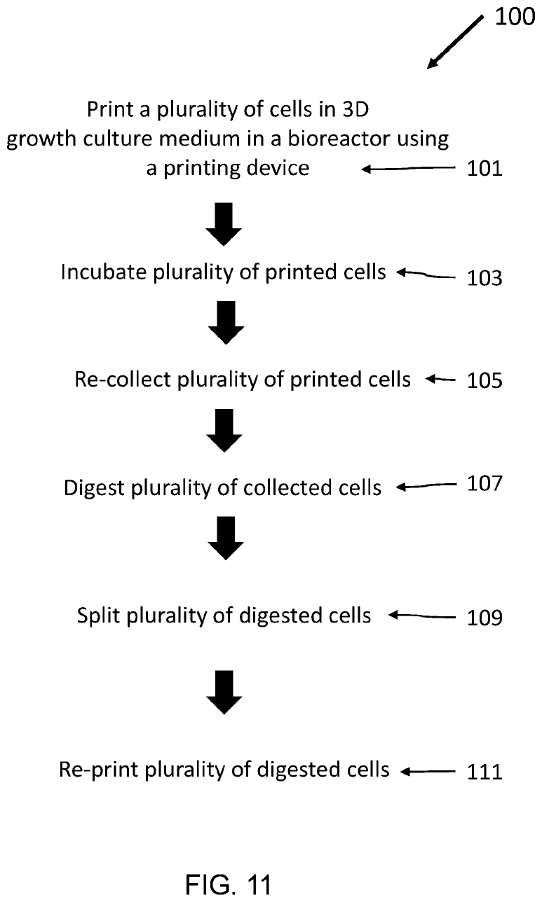

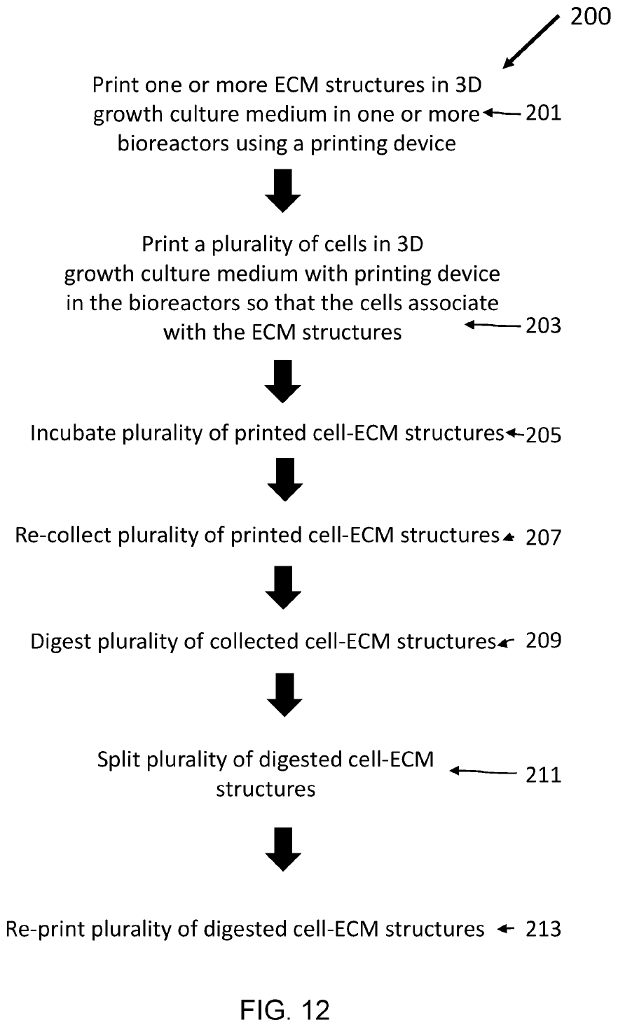

The flow charts of FIGS. 11-12 show examples of functionality that can be implemented in the apparatus 1010 of FIG. 10. If embodied in software, each block shown in FIGS. 11-12 can represent a module, segment, or portion of code that comprises program instructions to implement the specified logical function(s). The program instructions may be embodied in the form of source code that comprises machine code that comprises numerical instructions recognizable by a suitable execution system such as the processing device 202 (FIG. 10) in a computer system or other system. The machine code can be converted from the source code, etc. If embodied in hardware, each block may represent a circuit or a number of interconnected circuits to implement the specified logical function(s).

Although the flowcharts of FIGS. 11-12 show a specific order of execution, it is understood that the order of execution may differ from that which is depicted. For example, the order of execution of two or more blocks may be scrambled relative to the order shown. Also, two or more blocks shown in succession in FIGS. 11-12 can be executed concurrently or with partial concurrence. Further, in some embodiments, one or more of the blocks shown in FIGS. 11-12 can be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein that comprises software or code can be embodied in any non-transitory computer-readable medium for use by or in connection with an instruction execution system such as, for example, a processing device 202 in a computer system or other system. In this sense, each may comprise, for example, statements including instructions and declarations that can be fetched from the computer-readable medium and executed by the instruction execution system.

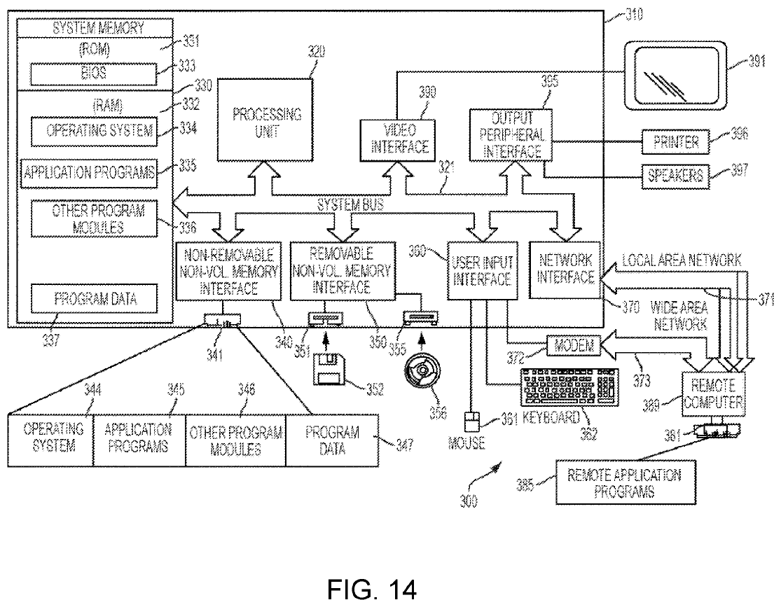

Techniques as described herein may be implemented on any suitable hardware, including a programmed computing system. A diagram of another embodiment of a computing system is shown in FIG. 14. For example, programming and printing the arrangement of cellular sheets as described herein may be performed by programming a computing device. Similarly, control of a 3D printing device to print biomaterials in accordance with a cellular sheet or method as described herein (for example FIGS. 11-12) may be controlled by a programmed computing device. FIG. 13 illustrates a system that may be implemented with multiple computing devices, which may be distributed and/or centralized. FIG. 14 illustrates an example of a suitable computing system environment 300 on which embodiments of these algorithms may be implemented. This computing system may be representative of a computing system that implements the techniques described herein. However, it should be appreciated that the computing system environment 300 is only one example of a suitable computing environment and is not intended to suggest any limitation as to the scope of use or functionality of aspects of the present disclosure. Neither should the computing environment 300 be interpreted as having any dependency or requirement relating to any one or combination of components illustrated in the exemplary operating environment 300.

Systems and methods described herein are operational with numerous other general purpose or special purpose computing system environments or configurations. Examples of well-known computing systems, environments, and/or configurations that may be suitable for use with the present disclosure include, but are not limited to, personal computers, server computers, hand-held or laptop devices, multiprocessor systems, microprocessor-based systems, set top boxes, programmable consumer electronics, network PCs, minicomputers, mainframe computers, distributed computing environments or cloud-based computing environments that include any of the above systems or devices, and the like.

The computing environment may execute computer-executable instructions, such as program modules. Generally, program modules include routines, programs, objects, components, data structures, etc. that perform particular tasks or implement particular abstract data types. Aspects of the present disclosure may also be practiced in distributed computing environments where tasks are performed by remote processing devices that are linked through a communications network. In a distributed computing environment, program modules may be located in both local and remote computer storage media including memory storage devices.

With reference to FIG. 14, an exemplary system for implementing aspects of the present disclosure includes a general-purpose computing device in the form of a computer 310. Though a programmed general-purpose computer is illustrated, it should be understood by one of skill in the art that algorithms may be implemented in any suitable computing device. Accordingly, techniques as described herein may be implemented in any suitable system. These techniques may be implemented in such network devices as originally manufactured or as a retrofit, such as by changing program memory devices holding programming for such network devices or software download. Thus, some or all of the components illustrated in FIG. 14, though illustrated as part of a general-purpose computer, may be regarded as representing portions of a node or other component in a network system.

Components of computer 310 may include, but are not limited to, a processing unit 320, a system memory 330, and a system bus 321 that couples various system components including the system memory 330 to the processing unit 320. The system bus 321 may be any of several types of bus structures including a memory bus or memory controller, a peripheral bus, and a local bus using any of a variety of bus architectures. By way of example and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 310 typically includes a variety of computer readable media. Computer readable media can be any available media that can be accessed by computer 310 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium that can be used to store the desired information and that can be accessed by computer 310. Communication media typically embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example and not limitation, communication media includes wired media such as a wired network or direct-wired connection, and wireless media such as acoustic, radio frequency (RF), infrared (IR), and other wireless media. Combinations of any of the above should also be included within the scope of computer readable media.

The system memory 330 includes computer storage media in the form of volatile and/or nonvolatile memory such as read only memory (ROM) 331 and random-access memory (RAM) 332. A basic input/output system 333 (BIOS), containing the basic routines that help to transfer information between elements within computer 310, such as during start-up, is typically stored in ROM 331. RAM 332 typically contains data and/or program modules that are immediately accessible to and/or presently being operated on by processing unit 320. By way of example and not limitation, FIG. 14 illustrates operating system 334, application programs 335, other program modules 336, and program data 337.

The computer 310 may also include other removable/non-removable, volatile/nonvolatile computer storage media. By way of example only, FIG. 14 illustrates a hard disk drive 341 that reads from or writes to non-removable, nonvolatile magnetic media, a magnetic disk drive 351 that reads from or writes to a removable, nonvolatile magnetic disk 352, and an optical disk drive 355 that reads from or writes to a removable, nonvolatile optical disk 356 such as a CD-ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 341 is typically connected to the system bus 321 through an non-removable memory interface such as interface 340, and magnetic disk drive 351 and optical disk drive 355 are typically connected to the system bus 321 by a removable memory interface, such as interface 350.

The drives and their associated computer storage media discussed above and illustrated in FIG. 14, provide storage of computer readable instructions, data structures, program modules, and other data for the computer 310. In FIG. 14, for example, hard disk drive 341 is illustrated as storing operating system 344, application programs 345, other program modules 346, and program data 347. Note that these components can either be the same as or different from operating system 334, application programs 335, other program modules 336, and program data 337. Operating system 344, application programs 345, other program modules 346, and program data 347 are given different numbers here to illustrate that, at a minimum, they are different copies. A user may enter commands and information into the computer 310 through input devices such as a keyboard 362 and pointing device 361, commonly referred to as a mouse, trackball, or touch pad. Other input devices (not shown) may include a microphone, joystick, game pad, satellite dish, scanner, or the like. These and other input devices are often connected to the processing unit 320 through a user input interface 360 that is coupled to the system bus, but may be connected by other interface and bus structures, such as a parallel port, game port, or a universal serial bus (USB). A monitor 391 or other type of display device is also connected to the system bus 321 via an interface, such as a video interface 390. In addition to the monitor, computers may also include other peripheral output devices such as speakers 397 and printer 396, which may be connected through an output peripheral interface 395.

The computer 310 may operate in a networked environment using logical connections to one or more remote computers, such as a remote computer 380. The remote computer 380 may be a personal computer, a server, a router, a network PC, a peer device, or some other common network node, and typically includes many or all of the elements described above relative to the computer 310, although only a memory storage device 381 has been illustrated in FIG. 14. The logical connections depicted in FIG. 14 include a local area network (LAN) 371 and a wide area network (WAN) 373, but may also include other networks. Such networking environments are commonplace in offices, enterprise-wide computer networks, intranets, and the Internet.

When used in a LAN networking environment, the computer 310 is connected to the LAN 371 through a network interface or adapter 370. When used in a WAN networking environment, the computer 310 typically includes a modem 372 or other means for establishing communications over the WAN 373, such as the Internet. The modem 372, which may be internal or external, may be connected to the system bus 321 via the user input interface 360, or other appropriate mechanism. In a networked environment, program modules depicted relative to the computer 310, or portions thereof, may be stored in the remote memory storage device. By way of example and not limitation, FIG. 14 illustrates remote application programs 385 as residing on memory device 381. It will be appreciated that the network connections shown are exemplary and other means of establishing a communications link between the computers may be used.

Having thus described several aspects of at least one embodiment of this present disclosure, it is to be appreciated that various alterations, modifications, and improvements will readily occur to those skilled in the art.

The above-described embodiments of the present disclosure can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software comprising logic that is executable on one or more processors that employ any one of a variety of operating systems or platforms. Additionally, such software or logic may be written using any of a number of suitable programming languages and/or programming or scripting tools, and also may be compiled as executable machine language code or intermediate code that is executed on a framework or virtual machine.

In this respect, aspects of the present disclosure may be embodied as a computer readable storage medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other tangible computer storage medium) encoded with one or more programs that, when executed on one or more computers or other processors, perform methods that implement the various embodiments of the present disclosure discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computers or other processors to implement various aspects of the present disclosure as discussed above. As used herein, the term “computer-readable storage medium” encompasses only a computer-readable medium that can be considered to be a manufacture (i.e., article of manufacture) or a machine. Alternatively, or additionally, aspects of the present disclosure may be embodied as a computer readable medium other than a computer-readable storage medium, such as a propagating signal.

The terms “program” or “software” are used herein in a generic sense to refer to any type of computer code or set of computer-executable instructions that can be employed to program a computer or other processor to implement various aspects of the present disclosure as discussed above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements

Cell-Culture Loop

3D growth medium made from packed microgels swollen in liquid culture media can enable the “cell culture loop” to be performed automatically, in which 3D cell-ECM structures are printed, incubated, re-collected, digested, diluted, and re-printed to expand populations.

Controlling the 3D flow distribution of liquid media through porous microgels can be an important factor for controlling cell viability and metabolism through diffusive transport into the 3D cell-ECM structures.

As described herein, a combination of media formulation, flow control, and ECM composition will enable the production of high-quality cells with the potential for industrial-scale up.

Described herein are methods of 3D culture. Methods of 3D culture as described herein can comprise the so-called “cell culture loop”: printing a plurality of cells into a bioreactor comprising a three-dimensional (3D) culture medium with a printing device into one or more sheets, wherein the 3D cell culture medium comprises a plurality of hydrogel particles and a liquid cell culture medium, wherein the hydrogel particles are swelled with the liquid cell culture medium to form a granular gel; incubating the plurality of printed cells in the bioreactor; re-collecting the incubated plurality of printed cells; digesting the collected plurality of printed cells; diluting the digested plurality of cells; and re-printing the diluted plurality of cells to expand populations. Methods of 3D culture as described herein can be manual methods, performed by a user with a syringe need, or can be automated methods, for example performed by a user operating a printing device operably connected to a computing device.

In embodiments, a sheet is a composition comprising a plurality of printed cells into a three-dimensional structure having a width less than the length or height. In embodiments, the length and/or width is greater than 10 times the thickness. In an embodiment, a sheet is rectangular in shape, although the skilled artisan would recognize that other geometric shapes are possible. In certain aspects, the sheets are printed along a printing axis that extends approximately orthogonally from the bottom of the reactor toward to the top opening of the bioreactor.

Sheets as described herein have a maximum thickness of about 400 microns. Such a thickness guarantees that any cell anywhere in the object is less than 200 microns away from a free surface (free surface that is or abuts the 3D cell growth medium). This maximum thickness is a hard upper limit because an object of the present disclosure is for the cells to proliferate and become dense or hit 70% density or confluence before passaging, which is the limit for dense tissue

Bioreactors as described herein can be perfusion-enabled bioreactors. Perfusion-enabled bioreactors as described herein allow for the flow of nutrient-containing liquid culture media to cells in the 3D culture media as described herein, while at the same time removing waste products away from the cells and space in the 3D growth media where cells are cultured. Bioreactors as described herein can be a plurality of bioreactors in an array, for example a multi-well culture plate.

Methods as described herein can utilize a printing device as described herein. A printing device can be a printing device with three orthongonal translation axes (x, y, z) that can moves a deposition needle or other disposition device around. Material deposition can be driven by volume displacement (e.g. with a syringe pump) or with controlled pressure from another pressure source, for example a vacuum.

Methods as described herein can further comprise printing one or more extra-cellular matrix (ECM) structures with the printing device in the bioreactor before incubating. In embodiments of the present disclosure, ECM structures as described herein are not self-supporting (i.e. require the 3D cell medium and/or cells for support). In embodiments according to the present disclosure, ECM structures as described herein are non-continuous. In embodiments according to the present disclosure, ECM structures as described herein comprise non-continuous biopolymer networks. In embodiments according to the present disclosure, ECM structures as described herein can comprise continuous biopolymer networks. In embodiments according to the present disclosure, ECM structures as described herein can comprise clusters of biopolymers. In embodiments according to the present disclosure, ECM structures as described herein can comprise aggregates of biopolymers.

Without intending to be limiting, in embodiments according to the present disclosure the one or more ECM structures (or components or constituents or biopolymers thereof) can comprise collagen-1, human placental matrix or components thereof, Matrigel®, laminin fibronectin. Constituents of ECM structures, such as collagen-1, human placental matrix or components thereof, Matrigel®, laminin fibronectin, and the like can be present in a concentration of about 0.1 mg/mL up to 30 mg/mL. In embodiments, within this concentration range ECM structures may be small biopolymer clusters or fragments or continuous biopolymer networks or gel-forming networks

In certain embodiments, the one or more ECM structures can be printed with cells in a sheet with a longest dimension extending from the bottom of the bioreactor to the top of the bioreactor. In embodiments according to the present disclosure, a sheet can be printed with a longest dimension approximately orthogonal to the bottom of the bioreactor.

In embodiments according to the present disclosure, the cells can be “doped” with 3D culture media or components thereof, and the plurality of cells can be mixed with 3D culture medium or hydrogels thereof before printing.

In embodiments according to the present disclosure, the one or more ECM structures can be “doped” with 3D culture media or components thereof, and the one or more ECM structures are mixed with 3D culture medium before printing.

The 3D culture medium can have a yield stress such that the cell growth medium undergoes a phase change from a first solid phase to a second liquid phase upon application of a shear stress greater than the yield stress.

The yield stress can be on the order of 10 Pa. The concentration of hydrogel particles can be between 0.05% to about 1.0% by weight. The hydrogel particles can have a size between about 0.1 μm to about 100 μm when swollen with the liquid cell culture medium.

The 3D culture medium in the bioreactor[s] can have a pore size of about 0.05 micron to about 5 micron.

The plurality of cells and/or one or more ECM structures can be disposed in a region of the 3D cell culture medium in the bioreactor[s].

The incubating can be for a time period of about 12 hours to about 240 hours. The incubating can be at a standard physiological temperature for the cells that are incubated, for example at about 37° C. The incubating can be a pO₂ of greater than about 10.5 kPa. In embodiments, the incubating can comprise gpCO2 at about 4.5 to about 6.0 kPa, pO2> about 10.5 kPa, and dissolved CO2 at about 23 mM to about 30 mM.

The re-collecting can be performed with a syringe needle having a bore diameter of about 0.05 mm to about 20 mm.

The syringe needle is operably connected to the printing device or extrusion head thereof.

The digesting can be an enzymatic digestion. Without intending to be limiting, in embodiments, the enzymatic digestion can comprise digestion with trypsin (or variant thereof), proteinase K, pepsin, or collagenase.

Cells can be split according to methods of the present disclosure. The splitting ratio according to the methods of the present disclosure can be a splitting ratio of about 1:2 to about 1:20. In embodiment according to the present disclosure, a 1:10 splitting ratio at 70% confluence (a standard in 2D culture) can be mimicked. In an embodiment according to the present disclosure, a 7% cell seeding density (volume fraction, ϕ) is employed when plating cells. In embodiments according to the present disclosure, structures at ϕ between 1% and 50% can be produced and printing mixtures with collagen (or other ECM components as described here) can be prepared at concentrations between about 0.25 mg/mL and about 5 mg/mL.

Systems

Also described herein are systems. Systems as described herein can comprise a printing system and 3D cell culture medium. Systems as described herein can comprise a printing system, a computing system, and 3D cell culture medium. Systems as described herein can comprise a printing system, a computing system, 3D cell culture medium, and one or more pluralities of cells. Systems as described herein can further comprise one or more bioreactors. Systems as described herein can further comprise ECM components for the printing of ECM structures.

Bioreactors

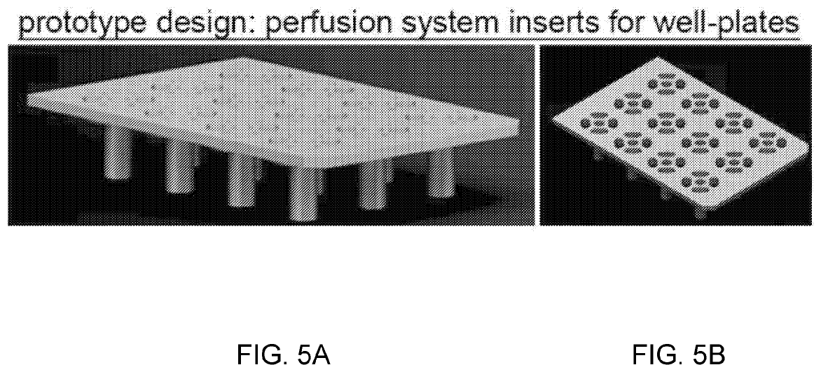

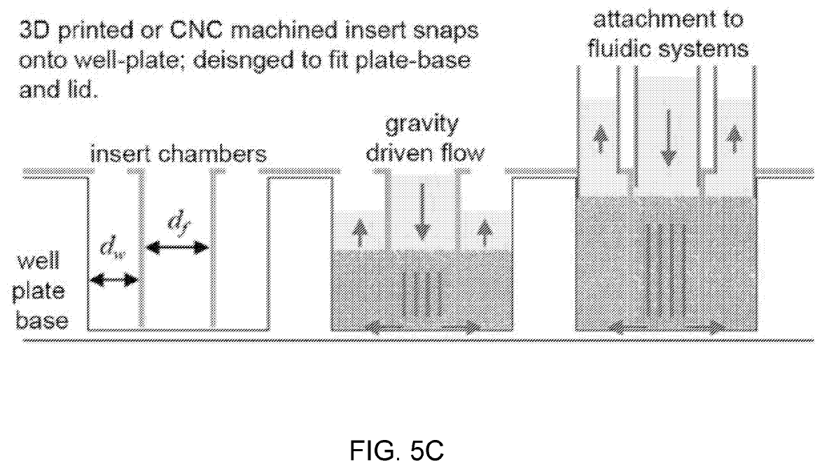

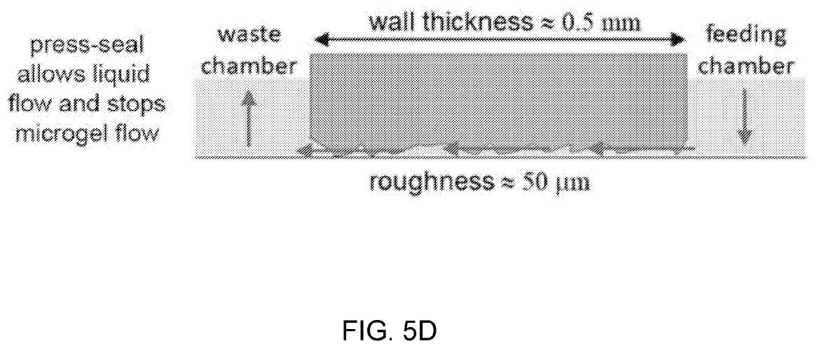

Bioreactors according to the present disclosure can be contained in standard multi-well tissue culture plates, of which a vast variety in various configurations are known in the art. Bioreactors according to the present disclosure can be perfusion-enabled, and can comprise an insert that provides perfusion functionality (embodiments of which are shown in FIGS. 5A-5B). Additional embodiments of perfusion-enabled bioreactors according to the present disclosure are such as those disclosed in International Application No. PCT/US2019/017316, the entire contents of which are incorporated by reference herein.

Kits

Further described herein are cell manufacturing kits. Cell manufacturing kits can comprise one or more bioreactors as described herein. Cell manufacturing kits can further comprise a plurality of cells and one or more ECM components. Cell manufacturing kits can further comprise 3D cell culture medium as described herein. In certain embodiments, at least the one or more ECM components and 3D cell culture medium can be supplied in the kit as a singular composition. Cells may be provided in the kit frozen with a cryoprotectant according to methods as known in the art.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

Other features, objects, and advantages of the present disclosure are apparent in the description that follows. It should be understood, however, that the description, while exemplifying certain embodiments of the present disclosure, is given by way of illustration only, not limitation. Various changes and modifications within the scope of the present disclosure will become apparent to those skilled in the art from the detailed description.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

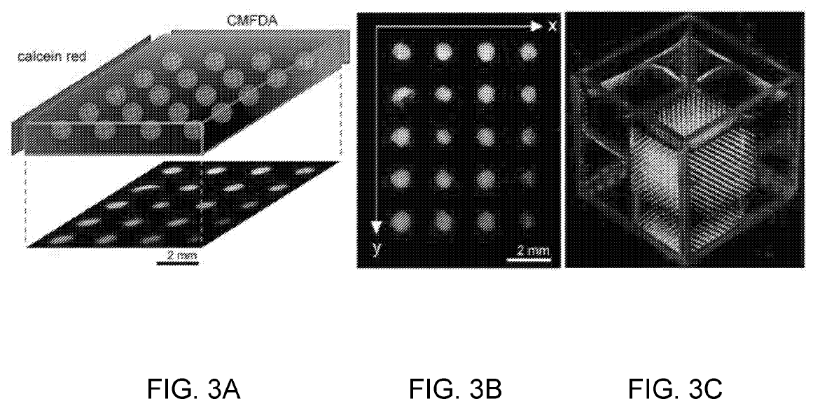

The combination of cells and packed microgel environments provides excellent control of variables like size, shape, symmetry, and topology in multicellular structures. Described herein are: (1) mechanical instabilities in simple structures to classify and measure collective cell forces; (2) the role of symmetry and topology of complex multicellular structures in collective cell dynamics. Research in area (1) has been completed and further research can be focused on area (2). Results from the research are significant because: (1) a superior platform for carrying out fundamental investigations of 3D cell dynamics was created; (2) a new class of biomaterial never before investigated was created; (3) fundamental aspects of collective cell dynamics previously inaccessible due to limitations of other support materials were explored (FIGS. 3A-3C).

As shown in FIGS. 3A-3C, time-release polymer films were loaded with fluorescent dyes that are activated by cellular metabolism once in the cytosol of HUH7 hepatocytes (green hues, CMFDA; red hues, calcein red-orange AM; FIG. 3A). A 2D gradient of dye uptake develops in spheroids made from HUH7 cells, where green signal is dominant in the upper right, red signal is dominant in the lower left, an intense mix of red and green is observed in the upper left, and the weakest combined signal is seen in the lower right (FIG. 3B). To show that large 3D printed arrays are achievable, 1000 spheres made from fluorescent colloids were printed in 1 h, shown here in a 2 in. quartz cube (FIG. 3C).

This new concept that breaks with established paradigms in cellular biomaterials is advancing knowledge in the areas of basic tissue mechanics, tissue culture methodology, and cellular biomaterials science and engineering.

### Example 2: Leveraging the Unique Properties of Packed Microgel 3D Printing Media to Design Cell-ECM Structures for Large-Scale Cell Production

In recent unpublished work the ability to control cell volume fraction (0-60%), collagen-1 concentration (0-5 mg/mL), and packed microgel concentration (0.5%-10% w/w) has been demonstrated. Control over the stability of printed structures by varying these parameters is also demonstrated. Numerous cell types have been printed including 3t3s and MSCs. According to embodiments of the present disclosure, the ability to print large, thin sheets, just 70 μm thick has been demonstrated. Finally, the ability to harvest these structures, dissociate the collagen, separate the cells, and re-print was demonstrated (FIGS. 4A-4C).

As shown in FIGS. 4A-4C, the fabrication and stability of high-aspect ratio structures made from cells and ECM (3t3 fibroblasts and collagen-1), printed into microgel medium, has been systematically explored (FIG. 4A). By varying the properties of the microgel culture medium, it can be controlled whether long, thin cylinders buckle, break up, contract axially, or remain static. Extremely thin macro-scale sheets (3D rendering of confocal fluorescence data, FIG. 4B) have also been printed, which demonstrate the ability to perform the proposed passaging steps (red 3t3+green collagen-1; FIG. 4C).

### Example 3: Leveraging the Unique Properties of Packed Microgel 3D Printing Media to Design Cell-ECM Structures for Large-Scale Cell Production

In current work on culturing 3D printed cell-ECM structures for extended durations, and in preparation for this proposal, perfusion systems were designed and tested that employ pressure-driven flow through packed microgel culture media (prototype system shown in FIGS. 5A-5D). Using microgels as a combined 3D printing environment and perfusable 3D culture medium provides a high level of flexibility in terms of design and operation of the tools and protocols for cell culture and experimentation. For example, the prototype system comprises a single piece of Delrin fabricated with a computer numerical control (CNC) machining system; Delrin can be autoclaved for sterilization. With this design, hollow cylinders are placed into wells containing microgel media; cell-ECM structures are printed and supplied with fresh liquid media inside of the cylinders and waste media is collected from the space between the outside of the cylinders and the walls of the culture wells (FIGS. 5A-5D). The cylinders press against the culture surfaces at the bottom of the wells, forming semi-permeable seals that allow liquid to flow between chambers but preventing microgels from passing from the feeding chambers into the waste chambers. 3D printing cell-ECM structures, feeding them, harvesting them, and collecting their waste is all performed though ports in the top surface of the plate insert. 3D printing instruments can be programmed to perform these tasks already, using gravity to drive liquid media through the feeding chamber and into the waste chamber. Results, for example in FIG. 6B, demonstrate control of microgel permeability, enabling perfusion sufficient to eliminate necrosis even in the centers of dense structures for 28 days; computational models can be used to interpret these observations (FIG. 6C).

As shown in FIGS. 5A-5D, perfusion chamber inserts were designed to snap-and-seal into 12-well plates, compatible with the plate lid (FIGS. 5A-5B). Within each well, the central chamber is used to 3D print cell-ECM structures and supply liquid growth media. The outer chamber is used to collect waste media (FIG. 5C). Preliminary tests show that gravity provides a sufficient pressure gradient to drive fluids from the feeding chamber to the waste chamber (FIG. 5C). Should controlled, continuous flow be necessary, pressurized perfusion tubes can be attached and sealed to the ports of the insert. The surface roughness of the fabricated structure can be chosen to determine fluid permeability while preventing microgel motion, trapping the gels in the percolated pore space between the base of the insert and the bottom of the well-plate (FIG. 5D).

As shown in FIGS. 6A-6C, the permeability, K, of microgel growth media is found by measuring the flow of a column of liquid growth media driven by gravity though a pack of length, L. The pore size in different microgel systems having particles of different diameter are estimated (FIG. 6A). Perfusion of 3D printed spheroids within microgel growth medium eliminates necrosis for up to 28 days (MG63 cells, hematoxylin and eosin stain; FIG. 6B). Models can predict the combinations of flowrate, cell metabolic rate, and molecular diffusivity required to support cells at a given volume fraction (FIG. 6C).

### Example 4: Develop Culture Conditions that Control Cell Phenotype, Gene Expression, and Stem Cell Differentiation in 3D

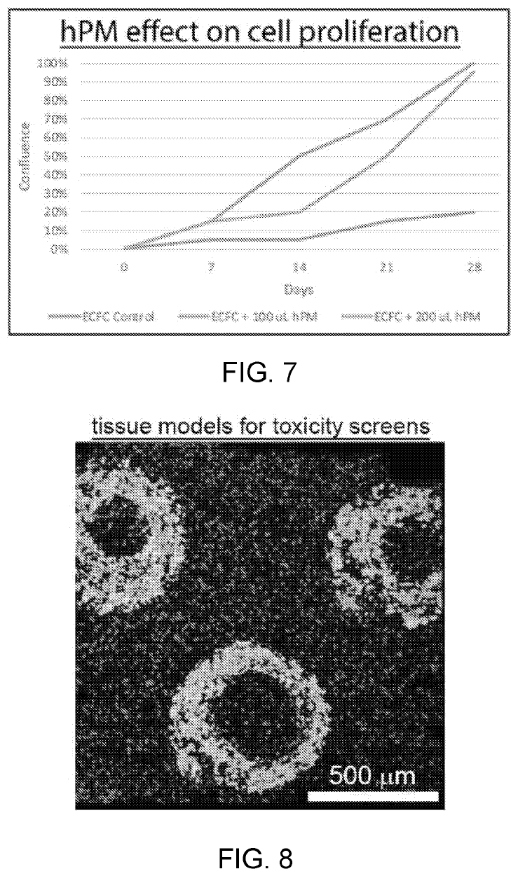

It was recently discovered that decellularized human placental matrix (hPM) has the remarkable ability to promote MSC proliferation without driving differentiation. hPM can be used to promote MSC growth if proliferation rates in 3D cell-ECM structures are found to be insufficient for cell production (FIG. 7).

FIG. 7 is a graft of human placental matrix (hPM) effect on cell proliferation. Endothelial colony forming cells (ECFCs), obtained from whole bone marrow density gradient separation, show the addition of hPM significantly increased proliferation without driving angiogenic differentiation.

### Example 5: 3D Printing of Small Liver Models

FIG. 8 is a photomicrograph demonstrating current work on drug-induced liver injury in 3D printed liver models in vitro, although this figure is not focused on cell manufacturing. Hepatocyte toroids (green) in a sea of fibroblasts (red), are supported by the 3D microgel medium.

### Example 6: Leveraging the Unique Properties of Packed Microgel 3D Printing Media to Design Cell-ECM Structures for Large-Scale Cell Production

1.1 Introduction.

The steps of cell expansion in 3D, as described here, are nearly the same as in 2D: seed, incubate, feed, harvest, disperse, dilute, and re-seed. Key differences for 3D are: (1) seeding is replaced by 3D printing; (2) feeding is done through perfusion. This process can be optimized for 3D with the objective of achieving control over each step of the “cell-culture loop”. As described herein, a 3D growth medium made from packed microgels swollen in liquid culture media can enable cells and ECM to be automatically structured in 3D space, incubated, re-collected, digested, diluted, and re-structured to expand populations. While the some clinically relevant cells include primary stem cells and induced pluripotent stem cells (iPSCs), other cells can be used, for example 3t3 fibroblasts. The 3t3 fibroblast cells are cost-effective and among the most studied immortalized cells; knowledge gained with 3t3s can serve as a foundation from which protocols can be optimized for working with other cells, such as with primary stem cells and iPSCs, with or without support from feeder cell populations. For similar reasons, bovine collagen 1 can be used as the ECM for this aim. As described herein, automated control of scaffold size, shape, cell seeding density, and ECM mesh-size throughout serial passages can be accomplished, up to passage numbers commensurate with methods and techniques of 2D culture or other practices.

1.2 Research Design

1.2.1 Optimizing 3D Printed High Aspect Ratio Cell-ECM Structures for Cell Culture.

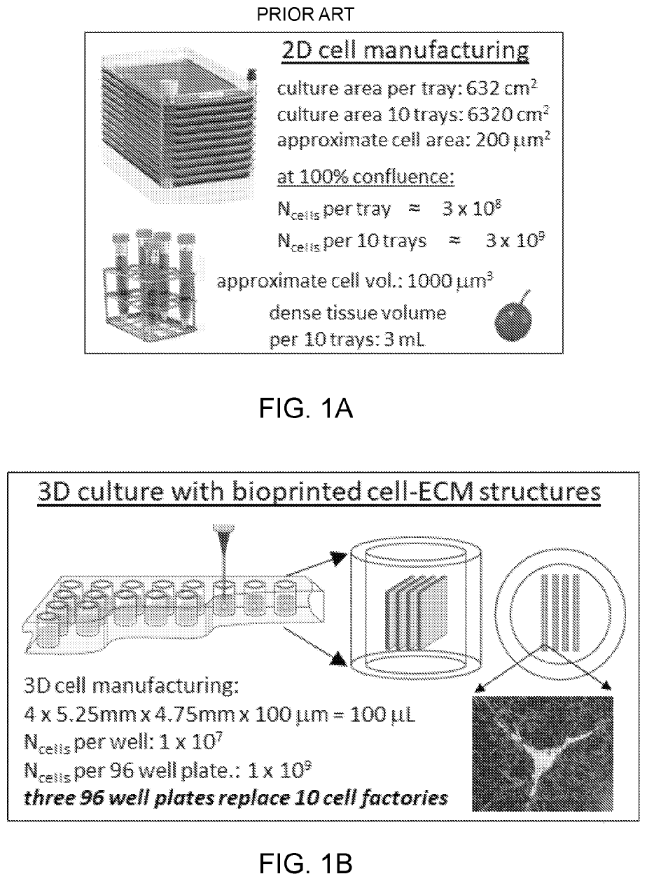

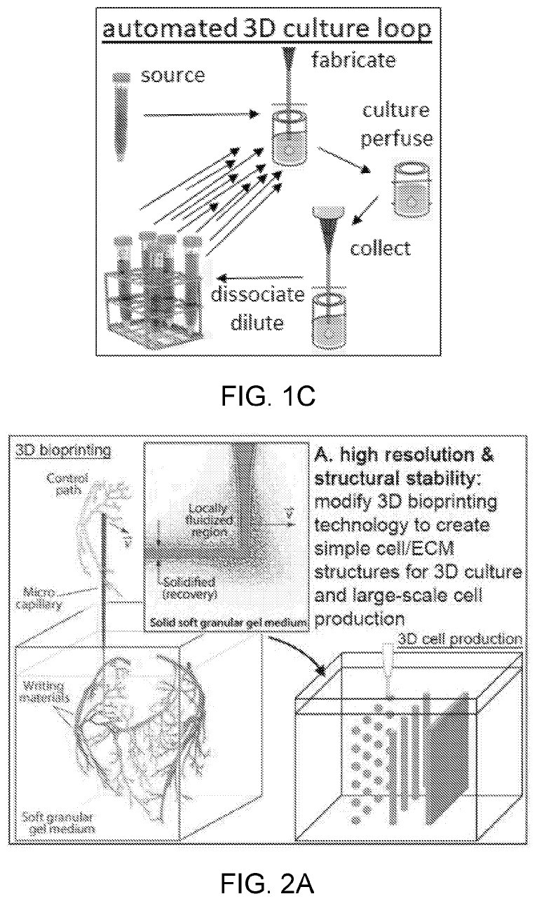

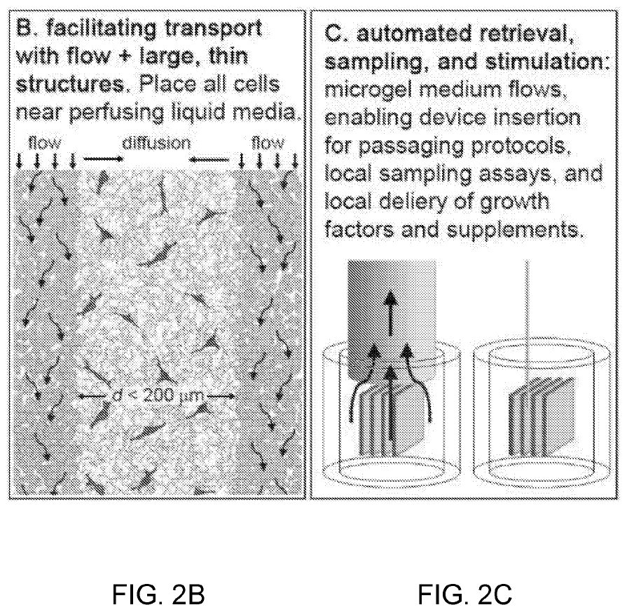

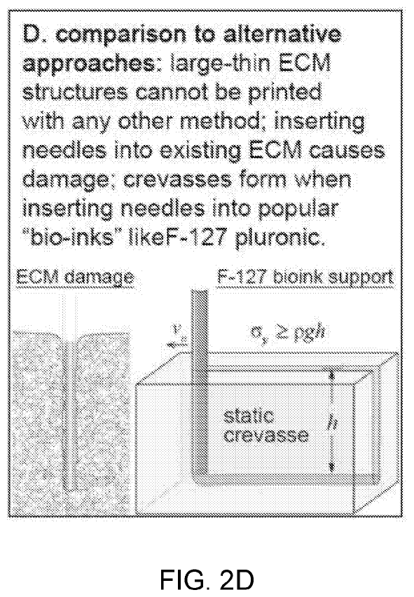

Creating cell-ECM structures that are thin along one dimension is important to ensure that diffusive transport from the surface to the core meets the metabolic needs of cells and removes waste products; when coupled to a perfusion system, large populations can be supported. There can be 3D printed large vertically oriented sheets made from 3t3 fibroblasts and collagen 1 (FIG. 1B, FIG. 2A-2D, FIG. 4A-4C), 0.5 cm tall, 1 cm wide, and varying in thickness (50-200 μm). This strategy embeds living cell populations between large sheet-shaped perfusable networks that can provide all the necessary molecular exchange between cells and feeding liquid. For the first generation of structures, cells can be grown in polystyrene culture flasks, harvested manually, and loaded into a syringe. The syringe needle can be inserted into the packed microgel culture medium contained within a glass-bottomed well-plate. In these studies, 12-well plates can be used, for example, to facilitate the design and fabrication of perfusion components, further described herein. Microgels currently synthesized in-house can be used, made from charge-neutral polyacrylamide. To mimic the 1:10 splitting ratio at 70% confluence (a standard in 2D culture), protocols as described herein can be optimized to achieve a 7% cell seeding density (volume fraction, ϕ). The bases of the cell-ECM structures can be reproducibly located less than 60 μm from the glass surface at the bottom of the well-plate; 3D confocal microscopy scans can be performed to measure cell population changes over the first 48 hours of growth. Cells can be fluorescently labelled with whole-cell dye (CMFDA). Cell number-density and volume will be measured with these confocal scans to estimate the cell volume fraction, ϕ, which is the 3D equivalent of “percent confluence” used in 2D. Additionally, confocal reflectance microscopy (CRM) can be performed to image the collagen fiber network and estimate its mesh-size, ξ, in the printed structures. To demonstrate control of ϕ and ξ, structures at ϕ between 1% and 50% can be produced and printing mixtures with collagen can be prepared at concentrations between 0.25 mg/mL and 5 mg/mL.

1.2.2 Harvesting Cell-Loaded ECM Scaffolds and Separating the Cells.

In ongoing work (see FIGS. 6A-6C, for example), the ability to manually lift cell-ECM structures from the microgel medium with a spatula or a micropipette for histological characterization has been demonstrated. These “tissue-like” objects are very dense. By contrast, for cell propagation, cell harvesting can be done before the cell-ECM structures become too dense, so it is expected that the structures to be very weak, potentially making a “lifting” procedure ineffective. New strategies for harvesting the cell-ECM structures can instead be employed, for example: (1) using a 3D printing system, locally visiting the structures with a narrow syringe needle and “vacuuming” them up one-at-a-time to minimize the amount of microgel retrieved; (2) fabricating a large-bore syringe needle, capable of collecting all structures within a well at once, along with a large amount of microgel material. Harvested material can be deposited into wells or centrifuge tubes for further processing. Harvested structures can be analyzed both before and after collagenase digestion of the ECM, using standard protocols; collagenase concentration and incubation times can be varied to maximize cell yield and minimize cell death. The proportion of cells collected relative to the initial population can be measured using both microscopy and whole-population fluorescence measurements using a plate-reader. Similarly, live-dead assays can be carried out using both methods to measure potential cell-death caused by the harvesting procedure. Cell death can be minimized and the proportion of harvested cells maximized by varying the diameter and translation speed of the collection-needle, and by varying the aspiration rate when collecting structures. Centrifugation to separate cells from supernatant liquid and any collected microgels can be performed. Stratification of liquid, microgels, and cells in centrifuged samples has been observed, enabling cell separation. Here, the harvesting steps can be performed automatically by a 3D printing instrument; the digestion and separation steps can be manual. The automation solutions capable of transferring centrifuge tubes between instruments already exists; building or buying such systems is too costly and unnecessary to develop the technology described herein. Success in this aspect of the disclosure can be assessed, for example, by comparing cell viability and efficiency of harvesting to the same metrics in 2D passaging.

1.2.3 3D Printing Serial Generations of Cells and Optimizing Splitting Ratios.

Before implementing and optimizing a perfusion system as described herein, serially passaging growing cell populations, in an embodiment, can be undertaken by performing the whole “cell culture loop” every two days on single sheet-shaped structures. This time-scale can allow a 24 h lag-time for cells to attach and re-establish their proliferation cycles, followed by one additional 24 h period in which all cells can be expected to divide. Starting with a ϕ=7% (volume fraction), it can be expected that ϕ at the 48-hour period can be at least 14%. A single large, thin sheet of cells approximately 1 cm×1 cm×100 μm in size can contain approximately 700,000 cells at a ϕ=7% (assuming a cell volume of 1000 μm³). Repeating this cycle, it is expected at least a doubling of the population can be seen with each passage; after 10 passages it is expected that approximately 7.2×10⁸ cells for each starting sheet will be produced. At each passaging step, the cell population growth can be measured using the methods described above or those known in the art. All passaged samples may not be necessary to be examined; for purposes as described herein 3-5 replicate structures can be reprinted in separate wells to enable statistical analysis of population growth. Splitting ratios can be varied at each passage to find the ratios that produce a population doubling every 48 hours. Once the perfusion system is in place and cells can be allowed to proliferate for 7-10 days, this aspect of the disclosure can be revisited to achieve an average population growth from ϕ=7% to 70% over this time-scale, as is standard in 2D for this cell type. The passaging cycle can be performed by following the 3D printing and harvesting protocols developed above; to perform the splitting and “re-printing” steps, harvested cells can be manually pipette-mixed with liquid growth medium and collagen-1 solution, then loaded into a sterile Hamilton gas-tight syringe, following current protocols. The loaded syringe can be mounted onto a printing system as described herein that can be spatially calibrated relative to the position of a well-plate to within ±30 μm in all three directions. Success in this aspect of the disclosure can be evaluated based on the ability to set 3D seeding density with serial passage for up to 10 passages.

While not all the steps described herein may necessarily be automated, steps that represent currently represent technological challenges can be automated in particular, although one of skill in the art would recognize that the degree of automation is not limited to this example. Such steps that can be automated include steps include 3D printing and harvesting of structures. The manual steps that can be performed, for example transferring to a centrifuge and separating cells from microgels and supernatant fluid, can be automated with currently available robotic systems.

### Additional Aspects of the Present Example

To eliminate potential issues during printing, such as cell sinking during printing, the cell-ECM mixture can be “doped” with microgels, gently trapping the cells in space within the syringe. This strategy can introduce microgels into the cell-ECM structures. However, it has been found that cells are able to push their way between microgels for division and migration; therefore this “doping” with microgels within the structure may not limit their growth. Furthermore, optimizing ECM concentration and splitting ratios that may be sensitive to the presence of microgels within the structures. Alternatively, to create large structures in just 10 s and eliminate all time constraints, flattened “cake frosting” shaped tips can be manufactured that allow for the extrusion of entire sheets in a single upward pass. To slow collagen gelation, the syringe barrels can be cooled with ice-packs or pelitier coolers when/if necessary.

### Example 7: Leveraging the Pore-Space in Microgel Packs to Exchange Nutrients and Waste with Large 3D Cell Populations Through Perfusion

2.1 Introduction.

To overcome the limitations of driving liquid through typical soft materials and making such materials perfusable, the pore-space existing between packed microgels can be leveraged to feed large populations of proliferating cells in 3D. As aspect of the present disclosure relates to the structural and operational parameters of perfusion systems that can support cell metabolism during incubation. In an embodiment, controlling the 3D flow distribution of liquid media through microgels is an important factor in controlling cell viability and metabolism through diffusive transport into the 3D cell-ECM structures. As aspect of the present disclosure can be to design and manufacture simple perfusion devices that insert into existing well plates. Justification for this approach is that it can facilitate: (1) confocal microscopy and plate-reader measurements; (2) modeling using known structural and geometric parameters (3) coupling to bioreactors and automation systems; (4) implementation in cGMP cell production facilities requiring sterility and quality control. By optimizing parameters relating to perfusion in systems and methods as described herein, control over cell health from an understanding of factors like pore-size, flow rate, and shape of cell-ECM structures can be realized.

2.2 Research Design

2.2.1 Explore Design and Operation of Well-Plate Perfusion Inserts.

With perfusion systems as described herein, ability to perfuse microgel packs within culture wells has been demonstrated, yet it is possible to explore a wider range of design and operation parameters to find conditions suitable for 3D cell production (see, for example, FIGS. 5A-5D and FIGS. 6A-6C). Aspects of the present disclosure described herein can be further explored by optimizing other parameters, such as varying microgel diameter (2 μm to 100 μm), which can control the length-scale of pore space (see, for example, FIGS. 6A-6C) and the global permeability. Additionally, different microgels can be used in the different chambers (feeding vs. waste), allowing simultaneous tuning of printing performance and system permeability. The chambers can be extended in height to control aspects of the present disclosure, including (but not limited to) the pressure-head, the printing volume, and the total volume of liquid exchanged during each feeding cycle. Initial tests using gravity to drive flow can comprise discrete feeding steps performed by a 3D printing instrument as described herein, similar to manual feeding. Here, global flow rates can be measured from changes in liquid column heights (FIGS. 6A-6C) and spatial variations in flow rates can be performed on an inverted confocal fluorescence microscope by injecting boluses of fluorescent dyes or by perfusing fluorospheres (40-400 nm diameter) smaller than the pores and throats between microgels. Continuous liquid exchange may be needed for large cell populations within small chamber volumes. Thus, the surface ports of the perfusion inserts can be designed to accommodate fittings typically used in perfusion bioreactors, and flow can be controlled, for example, using Masterflex® PC controllable peristaltic pumps and multi-chamber flow heads. Perfusion circuits can be coupled to analyzers such as, for example, a BioProfile® 400 Automated Chemistry Analyzer, to provide real-time, on-line monitoring of key nutrients, metabolites, and gases in cell culture media. Additional aspects of the present disclosure can be assessed, for example, by demonstrating: (1) the ability to control the average exchange rate of liquid media over a range sufficient to feed many different cell types (0.1-10 mL per 105 cells per day); (2) the ability to respond in real time to the changing metabolic demands of cells as populations rise.

2.2.2 Model Flow, Diffusion, Consumption.

Current computational modeling of large-scale tissue perfusion can be utilized to determine the sufficiency of glucose transport within 3D systems as described herein. Perfusion through packed microgels can depend on inlet and outlet conditions, varying dimensions of system structures, and dependent consumption rate of nutrients. Soft tissue biphasic models can provide the basis for analysis of deformation, flow, and nutrient distribution. A microgel pack can be modeled with the geometry defined by perfusion systems utilized (for example, those of FIGS. 5A-5D). These computational models can build upon previously developed interstitial transport models previously developed and transport analysis developed for perfusion bioreactors. Microgels and printed ECM can be represented by a biphasic model. Cell-ECM structures can be modeled as hyper-elastic, neo-Hookean materials. Fluid perfusion through microgels will be described by Darcy's law or the Brinkman formula. To account for the effect of cell growth on transport, the Carman-Kozeny relation may be used to predict changes in permeability with cell proliferation and compression. Glucose consumption can be treated as the limiting factor for cell metabolism and proliferation. Mass transport can be modeled as advection and diffusion through cell-ECM regions. Nutrient uptake by cells can be modeled by Michaelis-Menten kinetics. Changes in may be modeled accounting for cell migration, proliferation and death. Proliferation can be based on glucose availability by Contois kinetics. The models can combine governing biphasic, transport, and cell growth equations to solve for pore fluid pressure, velocity, shear stress, and solid matrix deformation and strain within the system space. These models can be developed using multiphysics software (such as, for example, COMSOL Inc., Burlington, Mass.). Modeling parameters can initially be abstracted from literature, as well as from separate mechanical and transport testing. Success can be assessed by comparing model predictions to measurements of glucose levels, cell viability, and metabolism, described above.

2.2.3 Measure Cell Health Under Different Flow Conditions.

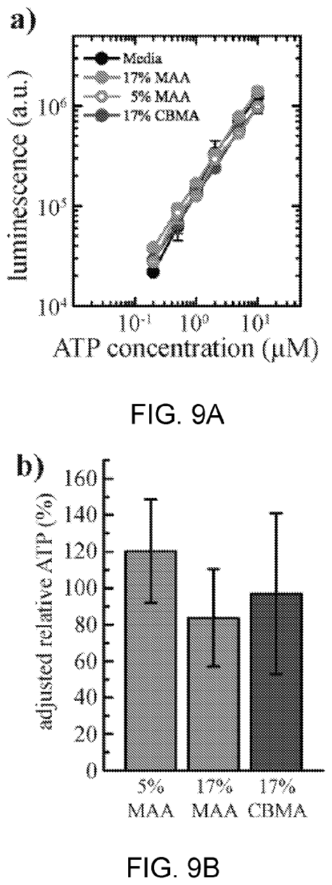

To measure cell health under different flow conditions, fluorescence-based live-dead assays can be performed and cell metabolic assays (CellTiter-Glo® and PrestoBlue®) in situ on intact structures using confocal microscopy and plate readers; the perfusion plate insert can be left in place or gently removed for the measurements. Additionally, structures can be removed from the microgel medium as described herein and the same assays can be performed in liquid media. Preliminary tests show that such assays can be compatible with microgel media; cells in 3D culture without perfusion after 24 hours exhibit the same viability and intracellular ATP as those in liquid culture (see, for example, FIGS. 9A-9B). Confocal microscopy measurements can provide spatial sensitivity while plate-reader assays provide quantitative averages. Using automated sampling and sensors for glucose, lactate, glutamine, glutamate, NH₄₊, pH, pO₂, pCO₂, sodium, potassium, with calculated osmolality, air saturation, and CO₂ saturation within flow circuit described herein, cell health can be monitored in real-time by comparing standard cultures to 3D systems and modulating inputs to meet cellular requirements. For example, low glucose (5.5 mM) can be suitable for MSC proliferative responses and high glucose (25 mM) induces differentiation. Auto-sampling coupled with automated nutrient input can allow for real-time control of critical media parameters; for example, lactate can manipulate glucose metabolism.

An aspect of the present disclosure can include an integrated perfusion bioreactor system that can produce healthy proliferating cells cultured in a 3D ECM micro-environment. This system can be designed with current tools and practices as described herein and others as known in the art in mind to facilitate adoption by industrial and academic labs while maintaining a small footprint, which is a major need for large-scale cell production. Designs as described herein can facilitate their manufacture on industrial scales; prototypes, for example, can be injection molded from polystyrene and sterilized using current practices. The microgel medium makes these goals possible by providing a high degree of flexibility that cannot be achieved with other 3D scaffolds. Results from computational modeling combined with optical and biochemical measurements can represent new knowledge of flow through porous biological media and its relationship to cellular metabolism and viability.

### Additional Aspects of the Present Disclosure

The microgel culture medium used here may not be confined on all sides; it can swell to a minimum packing density in the presence of excess liquid, reducing yield stress and making it susceptible to instability under flow-generated forces. Current formulations resist these instabilities during perfusion, though higher flow-rates may lead to the rearrangement of microgels and the resulting deformation of 3D printed cell-ECM structures. This problem may occur when cell volume-fraction is high and a rapid turnover of liquid media is needed. Solutions to this potential issue can include: (1) confining the microgels and the cell-ECM structures they support between semi-permeable nano-porous membranes like those used in trans-well culture plates; (2) manufacturing extra support walls or posts within well-plate inserts that provide rigid borders and stabilize microgel structures. These solutions can complicate the manufacture of perfusion systems, yet they are not prohibitive and employ approaches currently employed in mass production of cell culture tools (e.g. trans-well inserts). Additionally, it may be found that O₂ is the limiting metabolite; while some current models use glucose as the limiting nutrient. In this case, models can simply be modified using the currently accepted estimates of O₂ diffusivity and concentration in cell growth media and hydrogels.

### Example 8: Developing Culture Conditions that Control Phenotype, Gene Expression, and Stem Cell Pluripotency in 3D

3.1 Introduction.

To investigate the benefits of manufacturing cells in 3D ECM microenvironments using clinically relevant cells, an approach is to extend the aspects of the disclosure described above other cells, such as to stem cells, and to investigate gene expression profiles; here induced pluripotent stem cells (iPSCs), primary stem cells, and the 3t3s as a control can be studied. Such justification for this approach is that iPSCs and primary stem cells show great promise for growing the biomanufacturing industry in priority areas like cell therapies, tissue engineering, and regenerative medicine. The iPSCs can be generated from peripheral blood CD34+ cells as previously described by Hamazaki, T., El Rouby, N., Fredette, N. C., Santostefano, K. E. & Terada, N. Concise review: induced pluripotent stem cell research in the era of precision medicine. Stem Cells 35, 545-550 (2017) and Santostefano, K. E., Hamazaki, T., Biel, N. M., Jin, S., Umezawa, A. & Terada, N. A practical guide to induced pluripotent stem cell research using patient samples. Laboratory investigation 95, 4 (2015), both of which are incorporated by reference in their entireties as fully setforth herein. The ECM provided to iPSCs will be Matrigel®, laminin (such as iMatrix-511), or Collagen 1, including ROCK inhibitors such as Y-27632, as currently employed in 2D iPSC culture without feeder cells. Additionally, 3D suspension culture media for can be explored iPSCs (such as STEMCELL Tech, mTeSR-3D). Sources of primary stem cells can be, for example, commercially-available human perinatal stem cells purchased from vendors, also called Wharton's Jelly Stem Cells (WJSCs). WJSCs are among the few primary human stem cell lineages suitable for research purposes while having significant clinical potential; these cell lineage have been utilized and their differentiation directed in numerous applications including vascular, cartilage and bone regeneration. According to these aspects of the present disclosure, culture conditions producing cells exhibiting desired phenotype, gene expression profiles, and stem cell pluripotency metrics can be explored.

3.2 Research Design

3.2.1 Comparative Gene Expression Profiles in 2D Vs. 3D.

The outcomes according to aspects of the present disclosure as described herein can be used as starting points for working with the iPSCs and WJSCs studied in this example. Experimental conditions as described above can quickly allow for the establishment of baseline conditions for the maintenance and propagation of stem cells in 3D structures based on viability, metabolism, and ϕ. When these baseline conditions are established for stem cells, gene expression profiles can be measured. After 3D printing, cells can be assayed at the 24-hour, 1-day, and 10-day time points. After collection and separation, cells can be lysed using QIAzol® Lysis Reagent (Qiagen Sciences) following the manufacturer's protocol. Three (3) biological replicates can be used for each condition (2D and 3D culture). Cell lysates can be transported on dry ice to the Genomic Services Laboratory (HudsonAlpha) for RNA isolation and sequencing. Total RNA containing mRNA can be extracted from the cell lysate using the miRNeasy Kit with on-column DNase treatment (Qiagen). Polyadenylated RNAs can be isolated, for example, using NEBNext® Magnetic Oligo d(T)25 Beads. The NEBNext® mRNA Library Prep Reagent Set for Illumina (New England BioLabs®) can be used to prepare individually bar-coded next-generation sequencing expression libraries. Paired-end sequencing (25 million, 50-bp, paired-end reads) can be performed on an Illumina HiSeq2500 sequencer (Illumina). Post-processing of the sequencing reads from RNA-seq experiments for each sample can be performed using the Genomic Services Laboratory unique in-house RNA-seq data analysis pipeline. Raw reads can be mapped to the reference genome using TopHat v2.063. Samples can be grouped and transcript abundance quantified using Trimmed Means of M-values as the normalization method. Aspects of this example can be assessed by determining which genes change significantly in expression between 2D and 3D; differential expression of genes can be calculated on the basis of fold changes (using a default cut-off ≥±2.0) and the p-value of the differentially expressed gene list can estimate with z-score calculations using Benjamini Hochberg corrections of 0.05 for false-discovery rate using our previously published approach. Results can be represented on a volcano plot.

3.2.2 Gene Expression Profiles with Serial Passage.

The cell populations analyzed above may go through only one 3D passaging step when harvested; cell-ECM structures can be 3D printed into microgel media, incubated and perfused for the specified amount of time before harvesting for analysis. To measure potential accumulation of gene-expression irregularities with successive passages, the same gene-expression analysis described above can be repeated after additional passages (1, 4, and 9 subsequent passages, or other passage numbers), but this time for about 15 to 20 targeted genes that are found to be the most significantly altered in the global expression analysis described above. This can reduce project costs. Additionally, genes that are typically used to profile cells can be used, and their expression levels can be assayed with other techniques, such as Western Blotting or qRT-PCR analysis. Passaging can be performed when ϕ=70% is reached, using the approaches described in the previous sections. Additional aspects of this example can be assessed by comparing the expression of a chosen sub-set of genes at different passages with statistical confidence using, for example, the analysis methods described above.

3.2.3 Manipulate Stem-Cell Pluripotency and Cell Line Phenotype.

The gene-expression profiles investigated above can be collected under conditions found to achieve high levels of viability, proliferation rate, and metabolism in 3D, relative to their counterparts in 2D culture. These conditions may not be optimal for maintaining stem-cell pluripotency and cell-line phenotype; to achieve control aspects of the disclosure described above can be repeated under different conditions, varying media formulation using established cell re-programming cocktails, gas concentrations (pCO₂ at 4.5-6.0 kPa, pO2>10.5 kPa, and dissolved CO₂ at 23-30 mM), and the previously described factors like liquid media flow-rate and matrix composition. The gene-expression profiles can also be supplemented with in-house measurements using flow cytometry and immunofluorescence assays, targeting pluripotency markers like SSEA4, Nanog, Oct4, and Sox2 for stem cells; fibroblasts can be characterized by morphology and collagen 1 & 3 synthesis. Additionally, stem cells can be assayed using tri-lineage differentiation assays (STEMdiff™, STEMCELL Tech).

An aspect of the present disclosure can be the utilization of the culture conditions and approaches capable of producing high-quality cells with the desired properties for use in large-scale cell manufacturing—including clinically important cells like iPSCs and primary stem cells as well as immortalized cell lines for wide-spread use in basic research. The scientific outcome can include a systematically investigated map of the relationship between gene expression profiles and 3D culture conditions for multiple important cell types, enabled by the high level of control and automation provided by the novel 3D culture methods described herein.

### Additional Aspects of the Present Example

Promoting rapid stem cell proliferation while maintaining pluripotency can be challenging when placing cells in new microenvironments. Recent progress has demonstrated the capacity of decellularized human placental matrix (hPM) to promote stem-cell proliferation contexts while maintaining pluripotency (see FIG. 7). If iPSC and WJSC proliferation is insufficient to meet the needs of cell manufacturing, the potential enhancing proliferative effects of hPM on iPSCs and WJSCs can be utilized in 3D printed structures as described herein.

### Example 9

FIG. 11 is an embodiment of a method 100 according to the present disclosure. According to the method 100, a plurality of cells can be printed in 3D growth culture medium in a bioreactor using a printing device 101. The printed plurality of cells can be incubated 103. The incubated plurality of cells can be re-collected 105, digested 107, split 109, and re-printed 111 into 3D growth culture medium in a bioreactor.

### Example 10

FIG. 12 is an embodiment of a method 200 according to the present disclosure. According to the method 200, one or more ECM structures can be printed in 3D growth culture medium in one or more bioreactors using a printing device 201. A plurality of cells can be printed in 3D growth culture medium in one or more bioreactors using a printing device so that the cells associate with the ECM structures 203. The cells and ECM structures can be printed in one or more sheets. The printed cell-ECM structures can be incubated 205. In certain aspects, the printed cell-ECM structures can be incubated until each cell-ECM structure sheet reaches a density of about 70%. The incubated plurality of printed cell-EM structures can be re-collected 207, digested 209, split 211, and re-printed 213 into 3D growth culture medium in a bioreactor.

Unless defined otherwise, all technical and scientific terms used have the same meaning as commonly understood by one of ordinary skill in the art to which this disclosure belongs. Although any methods and materials similar or equivalent to those described can also be used in the practice or testing of the present disclosure, the preferred methods and materials are now described.

Embodiments of the present disclosure will employ, unless otherwise indicated, techniques of separating, testing, and constructing materials, which are within the skill of the art. Such techniques are explained fully in the literature.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

1. A method of 3D culture, comprising:
printing a composition comprising a plurality of cells into a sheet into a bioreactor with a printing device, wherein the bioreactor comprises a 3D cell culture medium comprising a plurality of hydrogel particles and a liquid cell culture medium, wherein the hydrogel particles are swelled with the liquid cell culture medium to form a granular gel;
incubating the sheet comprising the plurality of printed cells in the bioreactor;
re-collecting the incubated plurality of printed cells;
digesting the collected plurality of printed cells;
splitting the digested plurality of cells; and
re-printing the diluted plurality of cells in a second sheet to expand populations.

2. The method of claim 2, wherein the bioreactor is a perfusion-enabled bioreactor.

3. The method of claim 1, wherein the printing device comprises three orthogonal translation axes.

4. The method of claim 1, wherein printing the sheet further comprises printing one or more extra-cellular matrix (ECM) structures with the printing device in the bioreactor before incubating.

5. (canceled)

6. The method of claim 1, wherein the one or more ECM structures are printed in a sheet with a longest dimension extending from the bottom of the bioreactor to the top opening of the bioreactor.

7-8. (canceled)

9. The method of claim 1, wherein the 3D culture medium has a yield stress such that the cell growth medium undergoes a phase change from a first solid phase to a second liquid phase upon application of a shear stress greater than the yield stress.

10. The method of claim 9, wherein the yield stress is on the order of 10 Pa.

11. The method of claim 1, wherein the concentration of hydrogel particles is between 0.05% to about 1.0% by weight.

12. The method of claim 1, wherein the hydrogel particles have a size between about 0.1 μm to about 100 μm when swollen with the liquid cell culture medium.

13. The method of claim 1, wherein the 3D culture medium has a pore size of about 25 nm to about 25 μm.

14. The method of claim 1, wherein a plurality of cells are disposed in a region of the 3D cell culture medium.

15. The method of claim 1, wherein the incubating is for a time period of about 12 hours to about 240 hours.

16. The method of claim 1, wherein the incubating is at a temperature of about 30° C. to about 40° C.

17-18. (canceled)

19. The method of claim 1, wherein the syringe needle is operably connected to the printing device.

20-28. (canceled)

28. A cell manufacturing kit, comprising:
one or more bioreactors;
three-dimensional (3D) culture media;
a plurality of cells; and
one or more extracellular matrix components.

29. The cell manufacturing kit of claim 28, wherein the one or more bioreactors are perfusion-enabled bioreactors.

30. The cell manufacturing kit of claim 28, wherein the 3D culture media is a Herschel-Bulkley fluid.

31. The cell manufacturing kit of claim 30, wherein the 3D culture media has a yield stress of less than 100 pascals.

32. The cell manufacturing kit of claim 28, wherein the 3D culture media comprises a plurality of cross-linked polyacrylic acid polymers.

33-34. (canceled)

35. The cell manufacturing kit of claim 28, wherein the plurality of cells and one or more extracellular matrix components are provided as a sheet.

36-37. (canceled)
